SEQUENCE LISTING (7 sequences)

SEQ ID NO: 1 - DNA, 1429 bp, Homo sapiens
  source 1..1429: mol_type = genomic DNA
ggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcccagagcgcagaagctgggacgggccgtgggtcagagtgggcacgctgagcctacgccctcatgcggctggcccggccttggtccctagacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaccggcagtcctgccagctgttttggctagccga

SEQ ID NO: 2 - DNA, 1340 bp, Homo sapiens
  source 1..1340: mol_type = genomic DNA
ggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaccggcagtcctgccagctgttttggctagccga

SEQ ID NO: 3 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 4 - protein, 231 aa, Homo sapiens
MQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 5 - DNA, 1232 bp, Mus musculus
  source 1..1232: mol_type = genomic DNA
gatcgctgcgcgggctgcgagatctaggtggccgggcgcggagcccaagccgtgccgcgcggcgccatgaagggcaaggaggaaaaggagggcggcgcgcggctgggcactggtggcggcggcagccctgataagagcccgagtgcgcaagagctcaaggagcagggaaaccggctcttcgtgggccgcaagtacccggaggcggcggcctgctacggccgcgccatcactcggaacccacttgtggcagtgtactacactaaccgggccctgtgctatctgaagatgcagcagcctgaacaggcacttgctgactgccggcgagccctggagctggacgggcagtctgtgaaggcgcacttcttcctggggcagtgccagctggagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcattcatatctcaccaggctcattgctgctgagcgagagagggaactggaggagtgtcagcggaaccacgagggtcatgaagatgatggccacatccgggcccagcaggcctgcattgaggccaagcacgataaatacatggcagatatggatgagctcttctctcaggtggacgagaaaagaaagaagcgagatatccctgactacttgtgtggcaagattagctttgagctgatgcgggaaccctgcattacacccagtggtatcacctatgaccgcaaggacattgaggagcacctgcagcgtgtgggccactttgaccctgtgacccggagccctctgacccaggaacagctcatccccaacttggccatgaaggaagtcattgacgctttcatctctgagaacggctgggtagaggactattgaggccccatgtcctgcctggcaccctggcccaggaggatctggagacggaagctccagtccctgtatagtttgtgtccctgggcctgcccccatcggccctgctgatgggttctgaactgctccccttctcagcataccccttgctggaccatgagcctcccttgtcccccttctgggctggagagtgggtgagggtgggctgaggttgctgctgctgccactgtcctgtaataaagtctgtgagcactaca

SEQ ID NO: 6 - protein, 304 aa, Mus musculus
MKGKEEKEGGARLGTGGGGSPDKSPSAQELKEQGNRLFVGRKYPEAAACYGRAITRNPLVAVYYTNRALCYLKMQQPEQALADCRRALELDGQSVKAHFFLGQCQLEMESYDEAIANLQRAYSLAKEQRLNFGDDIPSALRIAKKKRWNSIEERRIHQESELHSYLTRLIAAERERELEECQRNHEGHEDDGHIRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 7 - protein, 8 aa, synthetic construct
SIINFEKL

(19) United States
(12) Patent Application Publication
(10) Pub. No.: US 2025/0352669 A1
(43) Pub. Date: Nov. 20, 2025

(54) METHODS AND COMPOSITIONS FOR TREATING CANCERS BY MODULATING THE EXPRESSION AND/OR ACTIVITY OF STUB1

(71) Applicant: President and Fellows of Harvard College, Cambridge, MA (US)

(72) Inventors: Martin W. LaFleur, Cambridge, MA (US); Arlene H. Sharpe, Cambridge, MA (US); Lauren E. Milling, Cambridge, MA (US); Nicole M. Derosia, Cambridge, MA (US); Ashlyn Michelle Lemmen, Cambridge, MA (US); Ivy Serondo Ling Streeter, Boston, MA (US); Qin Tjokrosurjo, Boston, MA (US); Paul Konstantin Simon Heisig, Cambridge, MA (US)

(73) Assignee: President and Fellows of Harvard College, Cambridge, MA (US)

(21) Appl. No.: 19/062,468

(22) Filed: Feb. 25, 2025

Related U.S. Application Data
(63) Continuation of application No. PCT/US2023/073178, filed on Aug. 30, 2023.
(60) Provisional application No. 63/402,176, filed on Aug. 30, 2022.

(51) Int. Cl. A61K 48/00; A61P 35/00; C12N 9/22; C12N 15/11; G01N 33/50

(52) U.S. Cl. CPC A61K 48/005; A61P 35/00; C12N 9/226; C12N 15/111; G01N 33/5023; G01N 33/5047; C12N 2310/20; G01N 2333/4703; G01N 2500/10

(57) ABSTRACT

The present invention provides methods and compositions for treating cancers, as well as methods for increasing an immune response against a tumor, in a subject in need thereof by decreasing the expression and/or activity of Stub1 in an immune cell of the subject.

### RELATED APPLICATION

This application is a continuation application of International Application No. PCT/US2023/073178, filed on Aug. 30, 2023, which claims the benefit of U.S. Provisional Application No. 63/402,176, filed on Aug. 30, 2022. The entire contents of each of the foregoing applications are incorporated herein by reference.

### SEQUENCE LISTING

The instant application contains a Sequence Listing which has been submitted electronically in XML format and is hereby incorporated by reference in its entirety. Said XML copy, created on Aug. 21, 2023, is named 117823-34520_SL.xml and is 11,731 bytes in size.

### BACKGROUND OF THE INVENTION

Cancer is one of the leading causes of death in industrialized nations. Cancers are caused by the progressive growth of the progeny of a single transformed cell. Treating cancer requires that malignant cells be removed or destroyed without killing the patient. An attractive way to achieve this would be to induce an immune response against the tumor that would discriminate between the cells of the tumor and their normal cell counterparts. Indeed, the immune system has a great potential for the specific destruction of tumors with no toxicity to normal tissue. The immune system's natural capacity to detect and destroy abnormal cells may prevent the development of many cancers. In addition, the long-term memory of the immune system may prevent cancer recurrence.

However, cancer cells are sometimes able to avoid detection and destruction by the immune system by reducing the expression of tumor antigens on their surface, making it harder for the immune system to detect them. Alternatively, cancer cells may express proteins on their surface that induce immune cell inactivation or induce cells in the surrounding environment to release substances that suppress immune responses and promote tumor cell proliferation and survival. Thus, there remains an ongoing and unmet need to identify new molecules that modulate the immune responses against tumors for the development of new therapeutic strategies to treat cancer.

### SUMMARY OF THE INVENTION

The present invention is based, at least in part, on the discovery that STUB1 plays a critical role in the suppression of the immune response to cancer. In particular, it has been discovered that Stub1 plays a negative regulatory role in immune responses against tumors, and reducing expression of Stub1 in immune cells, e.g., T cells, significantly attenuates tumor growth.

Accordingly, the present invention provides methods of treating a cancer or increasing an immune response to a cancer by decreasing or suppressing the expression and/or activity of Stub1 in immune cells, e.g., T cells.

In one aspect, the present invention provides a method of treating a cancer in a subject in need thereof, comprising decreasing the expression and/or activity of STIP1 Homology and U-Box Containing Protein 1 (Stub1) in an immune cell of the subject, thereby treating the cancer in the subject.

In some embodiments, the immune cell is selected from the group consisting of a T cell, a B cell, a natural killer (NK) cell, a dendritic cell, and a macrophage. In some embodiments, the immune cell comprises a T cell.

In some embodiments, the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

In some embodiments, the agent is a small molecule inhibitor of Stub1.

In some embodiments, the agent is a modified immune cell. In some embodiments, the modified immune cell is an immune cell derived from the subject modified to have a decreased level of expression and/or activity of Stub1.

In some embodiments, the modified immune cell having a decreased level of expression and/or activity of Stub1 is a modified T cell, e.g., a T cell modified to have a decreased level of expression and/or activity of Stub1. In some embodiments, the modified immune cell comprises a STUB1 knockout T cell.

In some embodiments, the modified immune cell having a decreased level of expression and/or activity of Stub1 further comprises a nucleic acid encoding a chimeric antigen receptor (CAR), wherein the CAR comprises an antigen binding domain, a transmembrane domain and an intracellular domain of a co-stimulatory molecule.

In some embodiments, the modified immune cell comprises a STUB1 knockout (KO) CAR T cell. In some embodiments, the STUB1 knockout CAR T cell is postive for IFNγ.

In some embodiments, the agent inhibits interaction between Stub1 and a binding partner.

In some embodiments, the binding partner is selected from the group consisting of E1 ubiquitin-activating enzymes, E2 ubiquitin-conjugating enzymes, heat shock protein family A member 4 (Hsp70), heat shock protein family A member 8 (Hsc70), forkhead box P3 (Foxp3), cysteine-rich hydrophobic domain 2 (Chic2), cardiomyocyte maturation associated lncRNA (CARMA), interferon gamma receptor 1 (IFNGR1), janus kinase 1 (Jak1), transforming growth factor (TGF)-beta, and a combination thereof.

In some embodiments, the subject is a human subject.

In some embodiments, the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In one aspect, the present invention provides a method of increasing an immune response against a tumor in a subject in need thereof, comprising decreasing the expression and/or activity of Stub1 in an immune cell of the subject, thereby increasing an immune response against the tumor in the subject.

In some embodiments, the immune cell is selected from the group consisting of a T cell, a B cell, a natural killer (NK) cell, a dendritic cell, and a macrophage. In some embodiments, the immune cell comprises a T cell.

In some embodiments, the method increases a T cell response.

In some embodiments, the method increases the number and/or activity of T cells.

In some embodiments, the T cells are CD8+ T cells.

In some embodiments, the method increases the expression of at least one marker selected from the group consisting of CD25, Granzyme B, TNFα, IFNγ, and Tim-3 in the immune cell.

In some embodiments, the method decreases the percentage of Slamf6-expressing immune cells. Alternatively or in addition, the method decreases expression of Slamf6 in the immune cell.

In some embodiments, the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

In some embodiments, the agent is a small molecule inhibitor of Stub1.

In some embodiments, the agent is a modified immune cell. In some embodiments, the modified immune cell is an immune cell derived from the subject modified to have a decreased level of expression and/or activity of Stub1.

In some embodiments, the modified immune cell having a decreased level of expression and/or activity of Stub1 is a modified T cell, e.g., a T cell modified to have a decreased level of expression and/or activity of Stub1. In some embodiments, the modified immune cell comprises a STUB1 knockout T cell.

In some embodiments, the modified immune cell having a decreased level of expression and/or activity of Stub1 further comprises a nucleic acid encoding a chimeric antigen receptor (CAR), wherein the CAR comprises an antigen binding domain, a transmembrane domain and an intracellular domain of a co-stimulatory molecule.

In some embodiments, the modified immune cell comprises a STUB1 knockout (KO) CAR T cell. In some embodiments, the STUB1 knockout CAR T cell is postive for IFNγ.

In some embodiments, the agent inhibits interaction between Stub1 and a binding partner.

In some embodiments, the binding partner is selected from the group consisting of E1 ubiquitin-activating enzymes, E2 ubiquitin-conjugating enzymes, heat shock protein family A member 4 (Hsp70), heat shock protein family A member 8 (Hsc70), forkhead box P3 (Foxp3), cysteine-rich hydrophobic domain 2 (Chic2), cardiomyocyte maturation associated lncRNA (CARMA), interferon gamma receptor 1 (IFNGR1), janus kinase 1 (Jak1), transforming growth factor (TGF)-beta, and a combination thereof.

In some embodiments, the subject is a human subject.

In some embodiments, the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In one aspect, the present invention provides a method of reducing a tumor size in a subject in need thereof, comprising decreasing the expression and/or activity of Stub1 in an immune cell of the subject, thereby reducing the tumor size in the subject.

In some embodiments, the immune cell is selected from the group consisting of a T cell, a B cell, a natural killer (NK) cell, a dendritic cell, and a macrophage. In some embodiments, the immune cell comprises a T cell.

In some embodiments, the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

In some embodiments, the agent is a small molecule inhibitor of Stub1.

In some embodiments, the agent is a modified immune cell. In some embodiments, the modified immune cell is an immune cell derived from the subject modified to have a decreased level of expression and/or activity of Stub1.

In some embodiments, the modified immune cell having a decreased level of expression and/or activity of Stub1 is a modified T cell, e.g., a T cell modified to have a decreased level of expression and/or activity of Stub1. In some embodiments, the modified immune cell comprises a STUB1 knockout T cell.

In some embodiments, the modified immune cell having a decreased level of expression and/or activity of Stub1 further comprises a nucleic acid encoding a chimeric antigen receptor (CAR), wherein the CAR comprises an antigen binding domain, a transmembrane domain and an intracellular domain of a co-stimulatory molecule.

In some embodiments, the modified immune cell comprises a STUB1 knockout (KO) CAR T cell. In some embodiments, the STUB1 knockout CAR T cell is postive for IFNγ.

In some embodiments, the agent inhibits interaction between Stub1 and a binding partner.

In some embodiments, the binding partner is selected from the group consisting of E1 ubiquitin-activating enzymes, E2 ubiquitin-conjugating enzymes, heat shock protein family A member 4 (Hsp70), heat shock protein family A member 8 (Hsc70), forkhead box P3 (Foxp3), cysteine-rich hydrophobic domain 2 (Chic2), cardiomyocyte maturation associated lncRNA (CARMA), interferon gamma receptor 1 (IFNGR1), janus kinase 1 (Jak1), transforming growth factor (TGF)-beta, and a combination thereof.

In some embodiments, the subject is a human subject.

In some embodiments, the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In one aspect, the present invention provides a method of prolonging the survival of a subject having a cancer, comprising decreasing the expression and/or activity of Stub1 in an immune cell of the subject, thereby prolonging the survival of the subject.

In some embodiments, the immune cell is selected from the group consisting of a T cell, a B cell, a natural killer (NK) cell, a dendritic cell, and a macrophage. In some embodiments, the immune cell comprises a T cell.

In some embodiments, the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

In some embodiments, the agent is a small molecule inhibitor of Stub1.

In some embodiments, the agent is a modified immune cell. In some embodiments, the modified immune cell is an immune cell derived from the subject modified to have a decreased level of expression and/or activity of Stub1.

In some embodiments, the modified immune cell having a decreased level of expression and/or activity of Stub1 is a modified T cell, e.g., a T cell modified to have a decreased level of expression and/or activity of Stub1. In some embodiments, the modified immune cell comprises a STUB1 knockout T cell.

In some embodiments, the modified immune cell having a decreased level of expression and/or activity of Stub1 further comprises a nucleic acid encoding a chimeric antigen receptor (CAR), wherein the CAR comprises an antigen binding domain, a transmembrane domain and an intracellular domain of a co-stimulatory molecule.

In some embodiments, the modified immune cell comprises a STUB1 knockout (KO) CAR T cell. In some embodiments, the STUB1 knockout CAR T cell is postive for IFNγ.

In some embodiments, the agent inhibits interaction between Stub1 and a binding partner.

In some embodiments, the binding partner is selected from the group consisting of E1 ubiquitin-activating enzymes, E2 ubiquitin-conjugating enzymes, heat shock protein family A member 4 (Hsp70), heat shock protein family A member 8 (Hsc70), forkhead box P3 (Foxp3), cysteine-rich hydrophobic domain 2 (Chic2), cardiomyocyte maturation associated lncRNA (CARMA), interferon gamma receptor 1 (IFNGR1), janus kinase 1 (Jak1), transforming growth factor (TGF)-beta and a combination thereof.

In some embodiments, the subject is a human subject.

In some embodiments, the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In some embodiments, the method increases the overall survival of the subject, and/or the progression-free survival of the subject. In some embodiments, the methods of the present invention further comprise administering to the subject an additional therapeutic treatment.

In some embodiments, the additional therapeutic treatment is selected from the group consisting of chemotherapy, endocrine therapy, antibody therapy, immunotherapy, cytokine therapy, growth factor therapy, hormone therapy, radiation therapy, surgery, or any combination thereof.

In some embodiments, the additional therapeutic treatment comprises an inhibitor of an immune-inhibitory protein, an immune checkpoint inhibitor, a chemotherapeutic agent, or a cytokine modulator.

In some embodiments, the additional therapeutic treatment comprises an inhibitor of cytotoxic T-lymphocyte-associated antigen 4 (CTLA4), an inhibitor of programmed cell death protein 1 (PD1), an inhibitor of programmed cell death protein 1 ligand (PDL1), an inhibitor of lymphocyte activation gene 3 (LAG3), an inhibitor of B7-H3, an inhibitor of B7-H4, or an inhibitor of T cell membrane protein 3 (TIM3), an inhibitor of T cell membrane protein 4 (TIM4), an inhibitor of V-Set Immunoregulatory Receptor (VISTA), an inhibitor of B7-H2, an inhibitor of B7-H6, an inhibitor of inducible T cell costimulatory (ICOS), an inhibitor of herpes virus entry mediator (HVEM), an inhibitor of CD160, an inhibitor of gp49B, an inhibitor of PIR-B, an inhibitor of KIR family receptors, an inhibitor of TIM-1, an inhibitor of B- and T-lymphocyte-associated protein (BTLA), an inhibitor of SlRPalpha (CD47), an inhibitor of CD48, an inhibitor of 2B4 (CD244), an inhibitor of B7.1, an inhibitor of B7.2, an inhibitor of leukocyte immunoglobulin like receptor B1 (ILT-2), an inhibitor of leukocyte immunoglobulin like receptor B2 (ILT-4), an inhibitor of T cell immunoreceptor with Ig and ITIM Domains (TIGIT), an inhibitor of HERV-H LTR-associating 2 (HHLA2), an inhibitor of butyrophilins, an inhibitor of adenosine A2a receptor (A2AR), an inhibitor of protein tyrosine phosphatase non-receptor type 2 (PTPN2), an inhibitor of CD39, an inhibitor of CD73, or an agonist antibody to GITR/OX40.

In one aspect, the present invention provides a method for identifying a compound useful for treating a cancer in a subject, comprising providing a test compound; determining the effect of the test compound on the expression and/or activity of Stub1; and selecting a compound which decreases the expression and/or activity of Stub1, thereby identifying a compound useful for treating a cancer in the subject.

In one aspect, the present invention provides a method of identifying a compound useful for increasing an immune response against a tumor in a subject in need thereof, comprising providing a test compound; determining the effect of the test compound on the expression and/or activity of Stub1 in an immune cell; and selecting a compound which decreases the expression and/or activity of Stub1, thereby identifying a compound useful for increasing an immune response against the tumor in the subject.

The present invention is illustrated by the following drawings and detailed description, which do not limit the scope of the invention described in the claims.

### BRIEF DESCRIPTION THE DRAWINGS

FIG. 1 depicts a 2747 gRNA screen that identified Stub1. To design this screen library, RNAseq was performed on naive and activated CD8+ T cells. Essential genes were filtered out essential genes, and genes with a GO term class associated with druggability were enriched. Pdcd1 and Ptpn2 were included for positive controls. A library with 2747 (899 genes targeted with 3 gRNAs/gene and 50 non-targeting/intergenic negative control gRNAs) was constructed. The screen was performed twice (left and right panels). The volcano plots were created by determining the change in gRNA abundance from the start (input) to tumor (output). These values were then z-scored to consider the negative control gRNAs to calculate the standard deviations away from the mean. Then, gRNAs were ranked from most enriched to most depleted and a p-value was calculated for multiple gRNAs per gene that were skewed (favoring enrichment or depletion) compared to the rest of the gRNAs using a hypergeometric test. The x-axis represents the z-scored effect sizes measured as enrichment or depletion of a gRNA relative to input. Note the absolute value of the z-score of 2 or greater is considered meaningful and a negative log 10 p-value of 3 or greater is considered highly significant. Individual gRNAs (3 per gene) are collapsed to gene level information on the plot. The Y-axis represents the average negative log 10 hypergeometric test calculated p-values, which measures the skew of the rank of the gRNAs for a given gene relative to the rest of the distribution. The top 5-6 enriched (positive x-axis values) and depleted (negative x-axis values) hits are indicated by gene identifier.

FIGS. 2A and 2B depict the log fold change results from the in vivo competitive assay in which nucleofection was used to KO genes in OT-1 CD8+ T cells to assess enrichment and depletion in tumors and lymph nodes, respectively. A 50:50 mix of control gRNA and each indicated gene was transferred into recipient mice that were subsequently implanted with B16 OVA tumors. Ten days later T cells were obtained from tumors for analyses. Y-axis was calculated by normalizing the output ratios to input ratios. The log 2 fold change of gRNA-targeted cells over control gRNA-targeted cells was calculated. X-axis represents the targeting gRNA (or control) in the competitive assay. Bars represent average and error bars represent standard deviation. Significance was calculated by one-way ANOVA comparing each group to the control group. (*p<0.05, **p<0.01, ***p<0.001, ****p<0.0001).

FIG. 3A depicts the B16 OVA tumor growth curves in wild-type mice that were recipients of 2,000 OT-1 T cells nucleofected with Stub1 or control gRNAs five days prior to implantation of 1 million B16 OVA tumor cells subcutaneously. The graph shows days post tumor injection (x-axis) and tumor volume (y-axis). Lines reflect average tumor growth across n=10 mice and error bars represent SEM. Significance of each comparison is denoted by line connecting the comparison groups. (*p<0.05, **p<0.01, ***p<0.001, ****p<0.0001). FIG. 3B depicts the survival curves showing days post tumor injection (x-axis) and the percentage of survival (y-axis). Significance of each comparison is denoted by line connecting the comparison groups. (**p<0.01).

FIG. 4A is a schematic depicting the generation of gene expression knockout in all hematopoietic lineages using a system that utilizes nucleofection to deliver Cas9-gRNA ribonucleoprotein complexes to cells. FIG. 4B depicts the percentage of insertion and deletion of Stub1 targeted by guide 1 (g1) and guide 2 (g2) in the immune system of indicated BMCs. FIG. 4C depicts the tumor growth curves showing days post tumor injection of 1 million MC38 tumor cells (x-axis) and tumor volume (y-axis), showing that KO of Stub1 in hematopoietic cells significantly controls tumor growth. Lines reflect average tumor growth across n=10 mice and error bars represent SEM. Clearance rate is denoted in the legend as number of mice with cleared tumors divided by total mice in the experiment. Significance is denoted by the line connecting the comparison groups. (*p<0.05, **p<0.01, ***p<0.001, ****p<0.0001). FIG. 4D depicts the survival curves showing days post tumor injection (x-axis) and the percentage of survival (y-axis). Significance of each comparison is denoted by line connecting the comparison groups. (****p<0.0001).

FIG. 5 depicts the log fold change results from the in vivo competitive assay in the LLC-OVA tumor model in tumor and lymph nodes. Y-axis was calculated by normalizing the output ratios to input ratios. Then, the log 2 fold change of gRNA-targeted OT-1 cells over control gRNA-targeted OT-1 cells was calculated. X-axis represents the targeting gRNA (or control) in the competitive assay. Bars represent average and error bars represent SD. Significance was calculated by one-way ANOVA comparing each group to the control group. (*p<0.05, **p<0.01, ***p<0.001, ****p<0.0001).

FIG. 6A depicts the representative overlaid flow cytometry histograms of cell trace violet (CTV) (x-axis) and modal-normalized counts (y-axis). Control cells are represented by the gray histogram and Stub1 KO cells are represented by the red histogram. Each peak represents a division and increasing CTV intensity indicates less division has occurred. FIG. 6B depicts the quantification of CTV divisions. X-axis represents the division number (higher numbers indicate more division has occurred). Y-axis represents the number of cells per division. Bars represent average and error bars represent SD. Significance was calculated by two-way ANOVA. (*p<0.05, **p<0.01, ***p<0.001, ****p<0.0001).

FIG. 7A depicts the quantification of the percentage of control or Stub1 KO OT-1 cells (Y-axis) for expression of the indicated markers on the X-axis in tumors and lymph nodes. Bars represent average and error bars represent SD. Significance was calculated by one-way ANOVA. (*p<0.05, **p<0.01).

FIG. 7B depicts the quantification of the percentage of control or Stub1 KO OT-1 cells (Y-axis) for expression of the indicated markers on the X-axis in tumors and lymph nodes. Bars represent average and error bars represent SD. DP represents double positive for tim3 and slamf6. DN represents double negative for tim3 and slam6. Significance was calculated by one-way ANOVA. (**p<0.01, ****p<0.0001).

FIG. 7C depicts the quantification of the percentage of control or Stub1 KO OT-1 cells (Y-axis) for expression of the IFNγ (left) and TNFα (right) in tumors. Bars represent average and error bars represent SD. Significance was calculated by one-way ANOVA. (*p<0.05, ****p<0.0001).

FIG. 7D depicts the number of control or Stub1 KO OT-1 cells (Y-axis) for expression of the indicated markers on the X-axis in tumors and lymph nodes. Bars represent average and error bars represent SD. DP represents double positive for tim3 and slamf6. DN represents double negative for tim3 and slam6. Significance was calculated by one-way ANOVA. (**p<0.01).

FIG. 8 depicts the log fold change (FC) results from the in vivo competitive assay in the B16-OVA model. Y-axis was calculated by normalizing the output ratios to input ratios. Then, the log 2 FC of gRNA-targeted OT-1 cells over control gRNA-targeted OT-1 cells was calculated. X-axis represents the organ assessed in the competitive assay. Bars represent average and error bars represent SD. Significance was calculated by ratio paired t-test. (*p<0.05, **p<0.01, ***p<0.001, ****p<0.0001).

FIG. 9A depicts the number of transferred control OT-1 cells recovered in B16 OVA tumors normalized to tumor size (in mg). X-axis represents the treatment group (isotype control or PD-1 blockade). Y-axis represents the number of control cells recovered normalized to tumor size. Lines represent average and individual symbols represent replicate animals. Significance was calculated by unpaired T-test. (*p<0.05, **p<0.01, ***p<0.001, ****p<0.0001).

FIG. 9B depicts the number of transferred control OT-1 cells recovered in lymph nodes. X-axis represents the treatment group (isotype control or PD-1 blockade). Y-axis represents the number of control cells recovered. Lines represent average and individual symbols represent replicate animals. Significance was calculated by unpaired t-test. (*p<0.05, **p<0.01, ***p<0.001, ****p<0.0001).

FIG. 9C depicts the log fold change results in B16 OVA tumors from the PD-1 blockade treated mice in the in vivo competitive assay. Y-axis was calculated by normalizing the output ratios to input ratios. Then, the log 2 fold change of gRNA-targeted OT-1 cells over control gRNA-targeted OT-1 cells was calculated. X-axis represents the group. Control is the control vs. control mix (black squares) and Stub1 G1 is the Stub1 G1 vs. control mix (magenta circles). Lines represent average and individual symbols represent replicate animals. Significance was calculated by two-way ANOVA. (*p<0.05, **p<0.01, ***p<0.001, ****p<0.0001).

FIG. 9D depicts the log fold change results in lymph nodes from the PD-1 blockade treated mice in the in vivo competitive assay described above. Y-axis was calculated by normalizing the output ratios to input ratios. Then, the log 2 fold change of gRNA-targeted OT-1 cells over control gRNA-targeted OT-1 cells was calculated. X-axis represents the group. Control is the control vs. control mix (black squares) and Stub1 G1 is the Stub1 G1 vs. control mix (magenta circles). Lines represent average and individual symbols represent replicate animals. Significance was calculated by two-way ANOVA. (*p<0.05, **p<0.01, ***p<0.001, ****p<0.0001).

FIG. 10A depicts the tumor growth curves in wild-type mice that were recipients of 2,000 OT-1 T cells nucleofected with Stub1 or control gRNAs five days prior to injection of Lewis lung carcinoma tumor cells subcutaneously. The graph shows days post tumor injection (x-axis) and tumor volume (y-axis). Lines reflect average tumor growth across n=10 mice and error bars represent SEM. Significance of each comparison is denoted by line connecting the comparison groups. (**p<0.01, ****p<0.0001). FIG. 10B depicts the survival curves showing days post tumor injection (x-axis) and the percentage of survival (y-axis). Significance of each comparison is denoted by line connecting the comparison groups. (*p<0.05, **p<0.01, ****p<0.0001).

FIG. 11 depicts the tumor growth curves showing days post tumor injection (x-axis) and tumor volume (y-axis), showing that KO of Stub1 in hematopoietic cells significantly controls B16-OVA tumor growth. Lines reflect average tumor growth across n=10 mice and error bars represent SEM. Clearance rate is denoted in the legend as number of mice with cleared tumors divided by total mice in the experiment. Significance is denoted by the line connecting the comparison groups. (*p<0.05, **p<0.01).

FIG. 12 depicts the tumor growth curves showing days post tumor injection (x-axis) and tumor volume (y-axis), showing that MC38 tumor growth control displayed by Stub1 KO BMCs is dependent on CD8+ T cells. CD8 Ab represents a CD8-depleting antibody and Isotype Ab represents an isotype control antibody. Lines reflect average tumor growth across n=10 mice and error bars represent SEM. Significance is denoted by the line connecting the comparison groups. (*p<0.05, ****p<0.0001).

FIG. 13 depicts the B16 OVA tumor growth curves showing days post tumor injection (x-axis) and tumor volume (y-axis), showing that IFNγ is required for the increased tumor growth control capacity of Stub1 KO T cells. Lines reflect average tumor growth across n=10 mice and error bars represent SEM. Significance is denoted by the line connecting the comparison groups. (*p<0.05, ***p<0.001).

### DETAILED DESCRIPTION OF THE INVENTION

The present invention is based, at least in part, on the discovery that Stub1 plays a critical role in suppression of the immune response to cancer. In particular, it has been discovered that Stub1 plays a negative regulatory role in immune responses against tumors, and reducing expression of Stub1 in immune cells, e.g., T cells, significantly attenuates tumor growth. Accordingly, the present invention provides methods of treating a cancer, methods of regulating an immune response to cancer, and methods of reducing tumor size and prolonging survival of subjects in need thereof by modulating, e.g., decreasing, the expression and/or activity of Stub1, in immune cells, e.g., T cells. In further embodiments, the invention includes methods for identifying a compound useful for treating a cancer and/or for increasing an immune response against a tumor in a subject in need thereof.

### I. Definitions

In order that the present invention may be more readily understood, certain terms are first defined. In addition, it should be noted that whenever a value or range of values of a parameter is recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

The term “comprising” or “comprises” is used herein in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein, the term “STUB1 homology and U-Box containing protein 1” or “Stub1” refers to a gene and protein also known as “C terminus of HSC70-Interacting Protein” (also known as CHIP; UBOX1; SCAR16; HSPABP2; NY-CO-7; SDCCAG7). This gene encodes a protein containing tetratricopeptide repeat and a U-box that functions as an E3 ubiquitin ligase/co-chaperone and promotes ubiquitination (also known as ubiquitylation). The sequence of a human STUB1 mRNA can be found, for example, at GenBank Accession GI: 1676324887 (NM_001293197.2; SEQ ID NO:1), or at GenBank Accession GI: 1519315992 (NM_005861.4; SEQ ID NO: 2). The sequence of a human Stub1 polypeptide sequence can be found, for example, at GenBank Accession No. GI: 56181387 (NP_005852.2; SEQ ID NO: 3), or at GenBank Accession No. GI: 645912990 (NP_001280126.1; SEQ ID NO:4). The sequence of a mouse STUB1 mRNA can be found, for example, at GenBank Accession GI: 1426138764 (NM_019719.4; SEQ ID NO:5). The sequence of a mouse Stub1 polypeptide sequence can be found, for example, at GenBank Accession No. GI: 9789907 (NP_062693.1; SEQ ID NO: 6).

As used herein, the term “immune cell” or “cells of the immune system” refers to any cells of the immune system involved in defending a subject against both infectious disease and foreign materials. Examples of immune cells include, without limitations, white blood cells including, e.g., neutrophils, eosinophils, basophils, lymphocytes (e.g., B-cells, T-cells, and natural killer cells), monocytes, macrophages (including, e.g., resident macrophages, resting macrophages, and activated macrophages); as well as Kupffer cells, histiocytes, dendritic cells, Langerhans cells, mast cells, microglia, and any combinations thereof. In some embodiment, immune cells include derived immune cells, for example, immune cells derived from lymphoid stem cells and/or myeloid stem cells. In some embodiment, immune cells include white blood cells (leukocytes) which are derived from hematopoietic stem cells (HSC) and/or hematopoietic progenitor cells (HPC). In some embodiment, immune cells include hematopoietic stem cells (HSC) and/or hematopoietic progenitor cells (HPC). In some embodiment, immune cells include lymphocytes (T cells, B cells, natural killer (NK) cells) and/or myeloid-derived cells (neutrophil, eosinophil, basophil, monocyte, macrophage, dendritic cells).

As used herein, the term “T cell” refers to all types of immune cells expressing CD3 including, without limitation, T-helper cells (CD4+ cells), cytotoxic T-cells (CD8+ cells), T-regulatory cells (Treg), and gamma-delta T cells. As used herein, the term “cytotoxic cell” refers, without limitation, to cells capable of mediating cytotoxicity responses, such as CD8+ T cells, natural-killer (NK) cells, and neutrophils.

The term “immune response” or “immune activity” as used herein refers to the action or interaction, including the end results, of one or more cells of the immune system (for example, T lymphocytes (e.g., effector T cells), B lymphocytes, natural killer (NK) cells, macrophages, eosinophils, mast cells, dendritic cells, neutrophils, and others) and soluble macromolecules produced by any of these cells (including antibodies, cytokines (e.g., IFN gamma, TNF alpha), chemokines (e.g., CXCL9, CXCL10), and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues. The term “immune activity” encompasses the activity or function of T cells, such as effector T cells as described herein, that is expressed towards a target cell (e.g., cancer cells) under both basal condition (non-immune challenge) and immune challenge or stimulation condition. In an embodiment, immune activity or immune response includes T cell-mediated and/or B cell-mediated immune responses that are influenced by modulation of T cell costimulation/co-inhibition. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., macrophages.

The term “effector T cell” as used herein refers to a naive T cell that has encountered antigen in the form of a peptide: MHC complex on the surface of an activated antigen-presenting cell (APC), and as a result, is induced to proliferate and differentiate into “effector T cells”. Effector T cells fall into two functional classes that detect different types of peptide:MHC complexes (including tumor antigens). For instance, peptides from intracellular pathogens that multiply in the cytoplasm are carried to the cell surface by MHC class I molecules and presented to CD8+ T cells. These differentiate into cytotoxic T cells that kill infected target cells. Peptide antigens from pathogens multiplying in intracellular vesicles, and those derived from ingested extracellular bacteria and toxins, are carried to the cell surface by MHC class II molecules and presented to CD4+ T cells. CD4+ T cells can differentiate into multiple types of effector T cells, including TH1+, TH2+, TH17+, and TFH cells, which help B cells become antibody-producing cells. (Immunobiology, 5th edition, The Immune System in Health and Disease (2001) by Charles A Janeway, Jr, Paul Travers, Mark Walport, and Mark J Shlomchik, New York: Garland Science; ISBN-10: 0-8153-3642-X). In some cases, extracellular antigens are presented on cells by MHC class I molecules. The presentation of internalized antigens on MHC I molecules is a process termed cross-presentation. Efficient cross-presentation has been shown to be crucial in, e.g., the induction of an adaptive immune response against tumors and viruses.

The term “effector T cell activity” as used herein refers to immune activity mediated by effector T cells upon signaling through the T cell receptor (TCR) expressed on T cells. In the context of the present invention, “effector T cell activity” encompasses the activity described above, for instance ability to induce apoptosis in a target cell by secreting perforin-granzymes as well as ability to kill or destroy pathogens or infected cells or aberrant cells (e.g., cancer cells displaying tumor antigens) by secreting substances such as cytokines (e.g. IFN gamma, TNF alpha) and chemokines (e.g. CXCL9, CXCL10).

As used herein, the term “an agent that decreases the expression and/or activity of Stub1” refers to an agent which directly or indirectly interferes with the expression and/or activity of STUB1, e.g., in an immune cell, e.g., a T cell. In some embodiments, the agents that decrease the expression and/or activity of Stub1 can modulate immune activity. Such agents may also be referred to as “modulator”. “Regulating,” “modifying” or “modulating” an immune activity refers to any alteration in a cell of the immune system (e.g., T cells such as effector T cells, cancer-infiltrating immune cells or other immune cells) or in the activity of such cell, for example as the consequence of such alteration. Such regulation includes stimulation or suppression or reduction of the immune activity which may be manifested by an increase or decrease in the number of various cell types, an increase or decrease in the activity of these cells (e.g., secretion of cytokines, chemokines, or perforin-granzymes), or increase or decrease in signaling pathway (e.g., PD-1/PD-L1 axis) between these cells, or any other changes which can occur within the immune system.

In some embodiments, the agent that decreases the expression and/or activity of Stub1 acts on the mRNA level, e.g., by regulating the expression and/or stability of the mRNA. In some embodiments, the agent that decreases the expression and/or activity of Stub1 acts on the protein level, e.g., by regulating the expression and/or stability of Stub1 protein, and/or the biological activity of Stub1.

In some embodiments, the agent that decreases the expression and/or activity of Stub1 comprises a modified immune cell or a modified hematopoietic cell. In some embodiments, the immune cells are derived from a subject, and are modified to have a decreased level of expression and/or activity of Stub1. In some embodiments, the level of expression and/or activity of Stub1 is decreased by contacting the immune cells with a nucleic acid capable of downregulating gene expression of STUB1. The nucleic acid capable of downregulating gene expression of STUB1 is selected from the group consisting of an antisense agent targeting STUB1, a double-stranded RNA agent targeting STUB1, an RNA-guided nuclease targeting STUB1, and a CRISPR system. In some embodiments, the modified immune cell having a decreased level of expression and/or activity of Stub1 further comprises a nucleic acid encoding a chimeric antigen receptor (CAR), wherein the CAR comprises an antigen binding domain, a transmembrane domain and an intracellular domain of a co-stimulatory molecule. In some embodiments, the modified immune cell comprises a T cell, a B cell, a natural killer (NK) cell, a dendritic cell, or a macrophage. In some embodiments, the agent that decreases the expression and/or activity of Stub1 is a modified T cell, e.g., a T cell having a decreased level of expression and/or activity of Stub1. In some embodiments, the agent that decreases the expression and/or activity of Stub1 is a STUB1 knockout T cell. In some embodiments, the STUB1 knockout T cell is postive for IFNγ. In some embodiments, the agent that decreases the expression and/or activity of Stub1 is a CAR T cell, wherein the expression of STUB1 is decreased or eliminated. In some embodiments, the agent that decreases the expression and/or activity of Stub1 is a STUB1 knockout CAR T cell. In some embodiments, the STUB1 knockout CAR T cell is postive for IFNγ.

In some embodiments, the agent that decreases the expression and/or activity of Stub1 comprises an antisense agent targeting STUB1, a double-stranded RNA agent targeting STUB1, an RNA-guided nuclease targeting STUB1, e.g., a CRISPR system, wherein the expression of STUB1 is decreased or eliminated.

In some embodiments, the agent that decreases the activity of Stub1 comprises a small molecule inhibitor of Stub1, an anti-Stub1 antibody or antigen-binding fragment thereof, a Stub1 fusion protein, or a Stub1 inhibitory peptide, wherein the activity of Stub1 is decreased or eliminated, e.g., in immune cells.

In one embodiment, the agent that decreases the expression and/or activity of Stub1 acts directly on Stub1, e.g., an antibody or a small molecule, by binding to Stub1 and decreases its activity or function. In another embodiment, the agent that decreases the expression and/or activity of Stub1 acts indirectly, e.g., through another molecule which interacts with Stub1, e.g., E1 ubiquitin-activating enzymes, or E2 ubiquitin-conjugating enzymes, thereby decreasing Stub1 expression and/or activity.

The phrase “contacting a cell with an agent,” such as an agent that decreases the expression and/or activity of Stub1, as used herein, includes contacting a cell by any possible means. Contacting a cell with an agent includes contacting a cell in vitro with the agent or contacting a cell in vivo with the agent. The contacting may be done directly or indirectly. Thus, for example, the agent may be put into physical contact with the cell by the individual performing the method, or alternatively, the agent may be put into a situation that will permit or cause it to subsequently come into contact with the cell.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In one embodiment, the subject is a mammal. In another embodiment, the subject is a human, such as a human being treated or assessed for cancer that would benefit from reduction in Stub1 expression and/or activity; a human at risk for developing cancer that would benefit from reduction in Stub1 expression and/or activity; a human having cancer that would benefit from reduction in Stub1 expression and/or activity; or human being treated for cancer that would benefit from reduction in Stub1 expression and/or activity. In some embodiments, the subject is a female human. In other embodiments, the subject is a male human. In some embodiments, the subject is a non-binary human. In one embodiment, the subject is an adult subject. In another embodiment, the subject is a pediatric subject.

As used herein, the terms “treating” or “treatment” refer to a beneficial or desired result, such as reducing at least one sign or symptom of a disease or disorder in a subject, for example, cancer. Treatment also includes a reduction of one or more sign or symptoms associated with Stub1 expression or a disease or disorder associated with Stub1 expression, e.g., cancer; diminishing the extent of Stub1 activation or stabilization or a disease or disorder associated with Stub1 expression, e.g., cancer; amelioration or palliation of Stub1 activation or stabilization or a disease or disorder associated with Stub1 expression, e.g., cancer. “Treatment” can also mean prolonging survival as compared to expected survival in the absence of treatment.

The terms “lower”, “decreased” or “reduced”, or related forms of such terms, in the context of the expression or activity of Stub1 in a subject or a disease marker or symptom refers to a statistically significant decrease in such expression or activity. The decrease can be, for example, at least 10%, 15%, 20%, 25%, 30%, 35%, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. In some embodiments, the terms “lower”, “decreased” or “reduced” also encompass eliminating the expression and/or activity of Stub1.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

### II. Methods of the Invention

The present invention is based, at least in part, on the discovery that Stub1 plays a critical role in suppression of immune responses to cancer. In particular, it has been discovered that Stub1 plays a negative regulatory role in immune response against tumors, and reducing expression of Stub1 in immune cells, e.g., T cells, significantly attenuated tumor growth.

Accordingly, the present invention provides methods of treating a cancer, methods of regulating an immune response to cancer, and methods of reducing tumor size and prolonging survival of subjects in need thereof by modulating, i.e., decreasing, the expression and/or activity of Stub1 in immune cells as well as its binding partners in the same pathway. In further embodiments, the invention includes methods for identifying a compound useful for treating a cancer and/or for increasing an immune response against a tumor in a subject in need thereof.

In one aspect, the present invention provides methods for increasing an immune response in a subject, by administering to the subject an effective amount of an agent that decreases the expression and/or activity of Stub1 in an immune cell of the subject. In one embodiment, decreasing the expression and/or activity of Stub1 in immune cells, e.g., T cells, results in an increase in the number, the abundance, or the proliferation of immune cells, e.g., T cells, e.g., CD4+ T cells and/or CD8+ T cells, in a subject. In another embodiment, decreasing the expression and/or activity of Stub1 in immune cells, e.g., T cells, results in an increase in the activity, e.g., activation and/or cytotoxicity, of immune cells, e.g., T cells, e.g., CD4+ T cells and/or CD8 T cells, in a subject. In a further embodiment, decreasing the expression and/or activity of Stub1 in immune cells, e.g., T cells, results in an increase in the expression of specific cell markers, e.g., CD25, Granzyme B, TNFα, IFNγ, and Tim-3, and a decrease in the percentage of Slamf6-expressing immune cells and/or decreases expression of Slamf6 in the immune cell.

The foregoing method can be used to treat a disorder which would benefit from increasing or augmenting the immune response in a subject. For example, an agent that decreases the expression and/or activity of Stub1 can be used to treat a cancer in a subject in need thereof. Such methods can include administering to a subject in need thereof, an effective amount of an agent that decreases the expression and/or activity levels of Stub1 in immune cells of a subject, thereby treating a cancer in the subject.

In another aspect, the present invention provides methods for reducing a tumor size in a subject in need thereof. The methods include administering to a subject in need thereof, an effective amount of an agent that decreases the expression and/or activity levels of Stub1 in immune cells of a subject, thereby reducing the tumor size in the subject.

In one aspect, the present invention features methods for prolonging the overall survival and/or progression-free survival of a subject in need thereof. The methods include administering to a subject in need thereof, an effective amount of an agent that decreases the expression and/or activity levels of Stub1 in immune cells of a subject, thereby prolonging the overall survival and/or progression-free survival of the subject.

In another aspect, the present invention features methods for increasing an immune response against a tumor in a subject in need thereof. The methods include administering to a subject in need thereof, an effective amount of an agent that decreases the expression and/or activity levels of Stub1 in immune cells of a subject, thereby increasing an immune response against the tumor in the subject.

The agents suitable for use in the methods of the present invention include any compound or molecule that can regulate the expression and/or activity of Stub1, for example, the mRNA expression and/or protein expression of Stub1; the mRNA and/or protein stability of Stub1; and/or the biological activity of Stub1. The agents can modulate the expression and/or activity of Stub1 either directly or indirectly. In some embodiment, the agent decreases the expression and/or activity of Stub1. The agents of Stub1 can act directly on Stub1, e.g., an antagonist antibody, or a small molecule inhibitor, which binds to Stub1 and decreases its activity or function. Alternatively, the agents could act indirectly on Stub1 (e.g., through another molecule, e.g., a binding partner of Stub1) resulting in a decreased activity. Exemplary modulators suitable for use in the methods of the invention include small molecule inhibitors, modified immune cells, modified hematopoietic cells, antagonist antibodies, or antigen-binding fragment thereof, inhibitory peptides, fusion proteins, or interfering nucleic acid molecules (e.g., antisense RNAs, dsRNAs, siRNAs, or an RNA-guided nuclease targeting STUB1). Modulators suitable for use in the methods of the invention are discussed in detail below.

The modulator of Stub1 of the present invention may also inhibit interaction between Stub1 and a binding partner. Binding partners of Stub1 can be identified by any methods known in the art. For example, protein-protein interaction between Stub1 and binding partners can be identified by co-immunoprecipitation in which the binding of a pair of proteins of interest is determined by forming a co-precipitate with a Stub1 antibody in vitro. Alternatively, the yeast two-hybrid or phage display approach may be employed to screen for binding partner of Stub1. In some embodiments, chemical cross-linking assays followed by mass spectrometry analysis can be used to identify interacting proteins.

Stub1 can function as a co-chaperone and an E3 ligase. As a co-chaperone, Stub1 can associate with heat-shock proteins and proteins targeted for degradation. In some embodiments, Stub1 interacts with Hsp70, Hsc70, and Foxp3 in CD4+T-regulatory cells and mediates degradation of Foxp3 in CD4+T-regulatory cells (Chen et al. Immunity 2013, 39(2)). As an E3 ligase, Stub1 associates with E1 ubiquitin-activating and E2 ubiquitin-conjugating enzymes to ubiquitin target proteins. Stub1 interacts with and ubiquitinates (non-degradative mark) CARMA in CD4+T lymphoblasts (Wang et al. European Journal of Immunology, 2013, 43(4): 1034-1041). Stub1 also interacts with IFNGR1, Jak1, and Hsp70 and mediates degradation of IFNGR1 and Jak1 in tumor cells (Apriamashvili et al. Nat Comm 2022, 23, 1923). Moreover, Stub1 interacts with and mediates degradation of TGF-beta in CD4+ T cells and MC38 tumor cells (Shen et al. Nat Comm 2022, 13, 3419). Lastly, Stub1 KO was shown to increase PD-L1 levels in cancer cells, suggesting that Stub1 and PD-L1 interact and that Stub1 mediates degradation of PD-L1 (Mezzadra et al. Nature 2017, 549, 7670).

The modulators of Stub1 suitable for use in the methods of the present invention may increase an immune response against a tumor by decreasing the activity involved in the Stub1 pathway, for example, by decreasing the expression and/or activity of any binding partners of Stub1.

The foregoing methods can be used to treat a disorder which would benefit from increasing or augmenting the immune response in a subject. Such disorders include, but are not limited to, cancer. Administration of an agent that decreases the expression and/or activity of Stub1 can be used, for example, to stimulate an immune response against a cancer (e.g., stimulate a T cell response against a cancer), reduce tumor size, and/or prolong survival, e.g., overall survival, and/or progression-free survival, of a subject having cancer.

As described herein, the term “cancer” refers to one of a group of diseases caused by the uncontrolled, abnormal proliferation of cells that can spread to adjoining tissues or other parts of the body. Cancer cells can form a solid tumor, in which the cancer cells are massed together, or exist as dispersed cells, as in leukemia. Types of cancer that are suitable to be treated by decreasing the expression or activity of Stub1 include, but are not limited to, solid tumors and/or hematological cancers. In one embodiment, the cancer is of epithelial origin. Exemplary types of cancer that can be treated by the foregoing methods include, but are not limited to, adrenal cancer, anal cancer, bile duct cancer, bladder cancer, bone cancer, brain/CNS tumors, breast cancer, castleman disease, cervical cancer, colon/rectum cancer, endometrial cancer, esophagus cancer, eye cancer, gallbladder cancer, gastrointestinal cancer, kidney cancer, laryngeal and hypopharyngeal cancer, leukemia, liver cancer, lung cancer, lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, waldenstrom macroglobulinemia, and Wilms tumor. In some embodiments, the cancer is selected from the group consisting of brain cancer, lung cancer, pancreatic cancer, melanoma, breast cancer, ovarian cancer, renal cell carcinoma, rectal adenocarcinoma, hepatocellular carcinoma, and Ewing sarcoma.

In some embodiments, the cancer is colon cancer, endometrioid cancer, kidney cancer (e.g., kidney papillary cell carcinoma, kidney clear cell carcinoma), liver cancer, thyroid cancer, lung cancer (e.g., lung adenocarcinoma, lung squamous cell carcinoma), head and neck cancer, breast cancer, cervical cancer, prostate cancer, bladder cancer, glioblastoma, rectal cancer, or bile duct cancer. In one embodiment, the cancer is brain cancer. In one embodiment, the cancer is glioblastoma. In one embodiment, the cancer is a blood-derived cancer. In one embodiment, the cancer is leukemia. In one embodiment, the cancer is acute myeloid leukemia (AML).

In some embodiments, the foregoing methods further comprise a screening step, wherein patients having a cancer are screened for expression and/or post-translation modifications of Stub1 in the immune cell, or in the cancer cell, or in the tumor microenvironment. For example, in one embodiment, a biological sample containing immune cells is obtained from the subject, and Stub1 expression is determined, and compared to a suitable control, e.g., a comparable sample obtained from a normal subject or a reference value indicative of the level of expression in a normal subject, etc. In another embodiment, the post-translation modification of Stub1 can be assessed in a biological sample containing immune cells obtained from the subject, and compared to a suitable control, e.g., a comparable sample obtained from a normal subject or a reference value indicative of the post-translation modification in a normal subject. In a further embodiment, the expression and/or post-translation modification of a relevant target, e.g. binding partner, of Stub1 is evaluated in a biological sample obtained from the subject, and compared to a suitable control, e.g., a comparable sample obtained from a normal subject or a reference value indicative of the level of expression and/or post-translation modification in a normal subject. Assessment of the expression and post-translation modification of STUB1, or a relevant target thereof, e.g., binding partner, may be performed either before and/or after administration of an agent to decrease expression and/or activity of STUB1 in an immune cell, in accordance with the methods of the present invention.

Pharmaceutical compositions described herein are suitable for administration in human or non-human subjects. Accordingly, the agent that decreases the expression and/or activity of Stub1, e.g., a small molecule inhibitor of Stub1, a modified immune cell having a decreased level of expression and/or activity of Stub1, e.g., a CAR T cell with a knockout for STUB1, and/or an anti-Stub1 antibody or antigen-binding portion thereof, described herein are useful as medicament for administering to a subject who is likely to benefit from reduced Stub1 expression and/or activity. In some embodiments, suitable subjects include healthy individuals who may nonetheless benefit from reduced Stub1 expression and/or activity. In some embodiments, suitable subjects have an existing cancer.

In some embodiments, suitable subjects are at risk of developing cancer. In some embodiments, suitable subjects are those who have previously had a surgery to remove tumor tissues. In some embodiments, suitable subjects are those on a therapy comprising another therapeutic agent to treat cancer, however, these therapies may be associated with adverse effects or high recurrence rates.

In some embodiments, such medicament is suitable for administration in a pediatric population, an adult population, and/or an elderly population.

The pediatric population in need for the agents decreasing the expression and/or activity of Stub1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, described herein may range between 0 and 6 months of age, between 0 and 12 months of age, between 0 and 18 months of age, between 0 and 24 months of age, between 0 and 36 months of age, between 0 and 72 months of age, between 6 and 36 months of age, between 6 and 36 months of age, between 6 and 72 months of age, between 12 and 36 months of age, between 12 and 72 months of age. In some embodiments, the pediatric population suitable for receiving the agents decreasing the expression and/or activity of Stub1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, described herein who is likely to benefit from such treatment may range between 0 and 6 years of age, between 0 and 12 years of age, between 3 and 12 years of age, between 0 and 17 years of age. In some embodiments, the population has an age of at least 5 years, e.g., 6, 7, 8, 9, 10, 11, 12, 13, 14, 15, 16, or 17 years. In some embodiments, the pediatric population may be aged below 18 years old. In some embodiments, the pediatric population may be (a) at least 5 years of age and (b) below 18 years of age.

The adult population in need for the agents decreasing the expression and/or activity of Stub1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, described herein may have an age of at least 18 years, e.g., at least 19, 20, 25, 30, 35, 40, 45, 50, 55, 60 or 65 years. In some embodiments, the adult population may be below 65 years of age. In some embodiments, the adult population may of (a) at least 18 years of age and (b) below 65 years of age.

The elderly population in need for the agents decreasing the expression and/or activity of Stub1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, described herein may have an age of 65 years or older (i.e., >65 years old), e.g., at least 70, 75 or 80 years.

A human subject who is likely to benefit from the treatment may be a human patient having, at risk of developing, or suspected of having cancer. A subject having cancer can be identified by routine medical examination, e.g., laboratory tests, biopsy, imaging tests, e.g., CT scans, MRI, or ultrasounds. A subject suspected of having any of such disease/disorder might show one or more symptoms of the disease/disorder. A subject at risk for the disease/disorder can be a subject having one or more of the risk factors for that disease/disorder.

A control subject, as described herein, is a subject who provides an appropriate reference for evaluating the effects of a particular treatment or intervention of a test subject or subject. Control subjects can be of similar age, race, gender, weight, height, and/or other features, or any combination thereof, to the test subjects.

In some embodiments, the agents that decrease the expression and/or activity of STUB1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, described herein are administered to a subject in need of the treatment at an amount sufficient to increase immune response, or reduce tumor growth, by at least 10% (e.g., 20% 30%, 40%, 50%, 60%, 70%, 80%, 90% or greater) in vivo. In other embodiments, the agents that decrease the expression and/or activity of Stub1, e.g., small molecule inhibitors of Stub1, modified immune cells having a decreased level of expression and/or activity of Stub1, e.g., CAR T cells with a knockout expression for STUB1, or anti-Stub1 antibodies and antigen-binding portions thereof, are administered in an amount effective to increase immune response, or reduce tumor growth by at least 10% (e.g., 20%, 30%, 40%, 50%, 60%, 70%, 80%, 90% or greater) in vitro. In other embodiments, agents that decrease the expression or activity of Stub1 are administered to increase T cell killing.

The particular dosage regimen, e.g., dose, timing and repetition, used in the method described herein will depend on the particular subject and that subject's medical history.

“An effective amount” as used herein refers to the amount of each active agent required to confer a therapeutic effect on the subject, either alone or in combination with one or more other active agents. For example, an effective amount refers to the amount of an agent decreasing the expression and/or activity of Stub1 of the present disclosure which is sufficient to achieve a biological effect, e.g., a decrease in the expression and/or activity of Stub1, or a reduction of tumor size.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, in the context of a decrease in the expression and/or activity of Stub1 in a cell, the decrease is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to a control expression and/or activity of Stub1. In one embodiment, the decrease in the expression and/or activity of Stub1 in the cell is a decrease in a range of 1-fold to 3-fold, 1.2-fold to 10-fold, 2-fold to 9-fold, 3-fold to 8-fold, 4-fold to 7-fold, 2-fold to 7-fold, etc. compared to the control expression and/or activity of Stub1.

In some embodiments, in the context of a decrease in the expression and/or activity of Stub1 in the cell after the administering step, the decrease is detectable within 4 hours, 24 hours, 48 hours, 7 days, 14 days, 21 days, 28 days or 30 days (or any time range bracketed by any of the listed duration of times) after the administering step. In one embodiment, a decrease in the expression and/or activity of Stub1 in the cell after the administering step is detectable for at least 5 days, 7 days, 14 days, 21 days, 28 days, or 30 days (or any time range bracketed by any of the listed duration of times) after the administering step. In one embodiment, a decrease in the expression and/or activity of Stub1 in the cell after the administering step is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to the expression and/or activity of Stub1 in the cell before the administering step. In one embodiment, a decrease in the expression and/or activity of Stub1 in the cell after the administering step is a decrease in a range of 1-fold to 3-fold, 1.2-fold to 10-fold, 2-fold to 9-fold, 3-fold to 8-fold, 4-fold to 7-fold, 2-fold to 7-fold, etc., compared to the expression and/or activity of Stub1 in the cell before the administering step.

In some embodiments, in the context of a decrease in the expression and/or activity of Stub1 in the immune cells after the administering step, a decrease is detectable within 4 hours, 24 hours, 48 hours, 7 days, 14 days, 21 days, 28 days, or 30 days (or any time range bracketed by any of the listed duration of times) after the administering step. In one embodiment, a decrease in the expression and/or activity of Stub1 in the immune cells after the administering step is detectable for at least 5 days, 7 days, 14 days, 21 days, 28 days, or 30 days (or any time range bracketed by any of the listed duration of times) after the administering step. In one embodiment, a decrease in the expression and/or activity of Stub1 in the immune cells after the administering step is at least 1-fold, 2-fold, 3-fold, 5-fold, 10-fold, 15-fold, 20-fold, 25-fold, 30-fold, 35-fold, 40-fold, 45-fold, or 50-fold or more (or any range bracketed by any of the values), compared to the expression and/or activity of Stub1 in the immune cells before the administering step. In one embodiment, a decrease in the expression and/or activity of Stub1 in the immune cells after the administering step is a decrease in a range of 1-fold to 3-fold, 1.2-fold to 10-fold, 2-fold to 9-fold, 3-fold to 8-fold, 4-fold to 7-fold, 2-fold to 7-fold, etc., compared to the expression and/or activity of Stub1 in the immune cells before the administering step.

Empirical considerations, such as the half-life, generally will contribute to the determination of the dosage. For example, antibodies and antigen-binding portions thereof that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a disease/disorder associated with Stub1, e.g., cancer. Alternatively, sustained continuous release formulations of an agent that decreases the expression and/or activity of Stub1, e.g., small molecule inhibitors of Stub1, or an anti-Stub1 antibody, or antigen-binding portion thereof, may be appropriate. Various formulations and devices for achieving sustained release would be apparent to the skilled artisan and are within the scope of this disclosure.

For the purpose of the present disclosure, the appropriate dosage of an agent that decreases the expression and/or activity of Stub1, will depend on the specific agent (or compositions thereof) employed, the type and severity of the disease/disorder, whether the agent is administered for preventive or therapeutic purposes, previous therapy, the patient's clinical history and response to the antagonist, and the discretion of the attending physician. In some embodiments, a clinician will administer an agent that decreases the expression and/or activity of Stub1, until a dosage is reached that achieves the desired result. Administration of an agent that decreases the expression and/or activity of Stub1 can be continuous or intermittent, depending, for example, upon the recipient's physiological condition, whether the purpose of the administration is therapeutic or prophylactic, and other factors known to skilled practitioners. The administration of an agent that decreases the expression and/or activity of Stub1 may be essentially continuous over a preselected period of time or may be in a series of spaced dose, e.g., either before, during, or after developing a disease or disorder associated with cancer.

The invention encompasses pharmaceutical compositions and related methods used as combination therapies for treating subjects who may benefit from reduction in Stub1 expression and/or activity in vivo. In any of these embodiments, such subjects may receive combination therapies that include a first composition comprising at least one agent that decreases the expression and/or activity of Stub1, e.g., small molecule inhibitors of Stub1, described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate. In some embodiments there are additive effects.

In some embodiments, combination therapies comprising a pharmaceutical composition described herein produce efficacy that is overall equivalent to that produced by another therapy (such as monotherapy of a second agent) but are associated with fewer unwanted adverse effects or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

Accordingly, the invention provides pharmaceutical compositions and methods for use in combination therapies for the treatment of cancer. In some embodiments, the methods or the pharmaceutical compositions further comprise a second therapy. The second therapy may diminish or treat at least one symptom(s) associated with the targeted disease. The first and second therapies may exert their biological effects by similar or unrelated mechanisms of action; or either one or both of the first and second therapies may exert their biological effects by a multiplicity of mechanisms of action.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

The one or more agents that decrease the expression and/or activity of Stub1 of the invention may be used in combination with one or more of additional therapeutic treatment. In some embodiments, the additional therapeutic treatment is selected from the group consisting of chemotherapy, endocrine therapy, antibody therapy, immunotherapy, cytokine therapy, growth factor therapy, hormone therapy, radiation therapy, surgery, and any combination thereof. In some embodiments, the additional therapeutic treatment which can be used with an agent of the invention include, but are not limited to, an inhibitor of an immune-inhibitory protein, an immune checkpoint inhibitor, chemotherapeutic agents, cytokine modulators, immunotherapeutic agents, immunosuppressive agents, and the like.

Such combination therapies may advantageously utilize lower dosages of the administered therapeutic agents, thus avoiding possible toxicities or complications associated with the various monotherapies.

Exemplary immune-inhibitory proteins include, but are not limited to cytotoxic T-lymphocyte-associated antigen 4 (CTLA4), programmed cell death protein 1 (PD1), programmed cell death protein 1 ligand (PDL1), lymphocyte activation gene 3 (LAG3), T cell membrane protein 3 (TIM3), T cell membrane protein 4 (TIM4), V-Set Immunoregulatory Receptor (VISTA), B7-H2, B7-H3, B7-H4, B7-H6, inducible T cell costimulatory (ICOS), herpes virus entry mediator (HVEM), CD160, gp49B, PIR-B, KIR family receptors, TIM-1, B- and T-lymphocyte-associated protein (BTLA), SlRPalpha (CD47), CD48, 2B4 (CD244), B7.1, B7.2, leukocyte immunoglobulin like receptor B1 (ILT-2), leukocyte immunoglobulin like receptor B2 (ILT-4), T cell immunoreceptor with Ig and ITIM Domains (TIGIT), HERV-H LTR-associating 2 (HHLA2), butyrophilins, CD39, CD73, and adenosine A2a receptor (A2AR). PD-1 is a checkpoint protein on T cells, which keeps T cells from attacking cells in the body that express PD-L1. Some cancer cells overexpress PD-L1, which enables them to evade detection by T cells, and inhibit T cell responses. Inhibitors of PD-L1 and PD-1 can boost the immune response against cancer cells, and can synergistically promote tumor cell killing when used in conjunction with agents that inhibit the expression and/or activity of STUB1. Exemplary anti-PD-L1 inhibitory antibodies include, but are not limited to, atezolizumab (Genentech), avelumab (Pfizer), and durvalumab (AstraZeneca). Exemplary anti-PD-1 inhibitory antibodies include, but are not limited to, pembrolizumab (Merck) and nivolumab (Bristol-Myers Squibb).

Chemotherapeutic agents include, for example, alkylating agents (e.g., cyclophosphamide, iphosphamide and the like), metabolism antagonists (e.g., methotrexate, 5-fluorouracil and the like), anticancer antibiotics (e.g., mitomycin, adriamycin and the like), vegetable-derived anticancer agents (e.g., vincristine, vindesine, taxol and the like), cisplatin, carboplatin, etoposide and the like. Among these substances, 5-fluorouracil derivatives such as furtulon and neofurtulon are preferred.

Exemplary cytokine modulators include, but are not limited to negative regulators of cytokines, e.g., protein tyrosine phosphatase non-receptor type 2 (PTPN2).

Immunotherapeutic agents include, for example, microorganisms or bacterial components (e.g., muramyl dipeptide derivative, picibanil and the like), polysaccharides having immune potentiating activity (e.g., lentinan, sizofilan, krestin and the like), cytokines obtained by a gene engineering technology (e.g., interferon, interleukin (IL) and the like), colony stimulating factors (e.g., granulocyte colony stimulating factor, erythropoetin and the like) and the like, among these substances, those preferred are IL-1, IL-2, IL-12 and the like.

Immunosuppressive agents may be used as a conditioning treatment for the agents of the invention, e.g., Stub1 KO CAR T cells, and may include, for example, calcineurin inhibitor/immunophilin modulators such as cyclosporine (Sandimmune, Gengraf, Neoral), tacrolimus (Prograf, FK506), ASM 981, sirolimus (RAPA, rapamycin, Rapamune), or its derivative SDZ-RAD, glucocorticoids (prednisone, prednisolone, methylprednisolone, dexamethasone and the like), purine synthesis inhibitors (mycophenolate mofetil, MMF, CellCept®, azathioprine, cyclophosphamide), interleukin antagonists (basiliximab, daclizumab, deoxyspergualin), lymphocyte-depleting agents such as antithymocyte globulin (Thymoglobulin, Lymphoglobuline), anti-CD3 antibody (OKT3), and the like.

Any of the above-mentioned agents can be administered in combination with the agent that decreases the expression and/or activity of Stub1 to treat cancer.

### III. Agents that Decrease the Expression and/or Activity of Stub1

As described above, the invention includes, in some embodiments, agents that decrease the expression and/or activity of Stub1 in immune cells in order to increase immune response against a tumor. Examples of such agents that may be used in the methods and compositions described herein are provided below, and include, but are not limited to, small molecule inhibitors, modified immune cells, modified hematopoietic cells, antagonist antibodies, or antigen-binding fragment thereof, inhibitory peptides, fusion proteins, or interfering nucleic acid molecules (e.g., antisense RNAs, dsRNAs, siRNAs, or an RNA-guided nuclease targeting STUB1). In some embodiments, the agent functions at a level of transcription and mRNA stability. In other embodiments, the agent acts at a level of translation, protein stability/degradation, protein modification, and protein binding.

### Inhibitory Nucleic Acids

In one embodiment, the methods described herein include targeting STUB1 using inhibitory nucleic acids. A nucleic acid inhibitor can encode a small interference RNA (e.g., an RNAi agent) that targets the STUB1 gene, or a gene encoding for another protein that interacts with Stub1, and decreases the expression or activity. The term “RNAi agent” refers to an RNA, or analog thereof, having sufficient sequence complementarity to a target RNA to direct RNA interference. Examples also include a DNA that can be used to make the RNA.

RNA Interference: RNA interference (RNAi) refers to a sequence-specific or selective process by which a target molecule (e.g., a target gene, protein or RNA) is down-regulated. Generally, an interfering RNA (“RNAi”) is a double stranded short-interfering RNA (siRNA), short hairpin RNA (shRNA), or single-stranded micro-RNA (miRNA) that results in catalytic degradation of specific mRNAs, and also can be used to lower or inhibit gene expression. RNA interference (RNAi) is a process whereby double-stranded RNA (dsRNA) induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev., 12: 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other dsRNAs which are expressed in vivo using DNA templates with RNA polymerase II or III promoters (Zeng et al., Mol. Cell 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002).)

siRNA Molecules: The term “short interfering RNA” or “siRNA” (also known as “small interfering RNAs”) refers to an RNA agent, preferably a double-stranded agent, of about 10-50 nucleotides in length, preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery.

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but are not limited to, 2′-O-methyl, 2′-fluoro, 2′-O-methyoxyethyl and phosphorothioate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotopes.

siRNA Delivery: Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g., stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol. Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007)).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression: Synthetic siRNAs can be delivered into cells, e.g., by direct delivery, cationic liposome transfection, and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol II and III promoter systems (e.g., H1, U1, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al., Genes & Dev. 20:515-524 (2006)). One common feature of miRNAs is that they are excised from an approximately 70 nucleotide precursor RNA stem loop by Dicer, an RNase III enzyme, or a homolog thereof. By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Antisense: An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, e.g., sequences relating to STUB1, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for decrease or inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit, reduce, or decrease the expression of the protein, e.g., by inhibiting, reducing, or decreasing transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited or decreased by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays. 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

### Fusion Proteins and RNA-Guided Nucleases

In another aspect of the invention, the agent that decreases the expression and/or activity of Stub1 in immune cells is a fusion protein.

As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

In some embodiments, the fusion proteins of the invention include Stub1 fused to an effector molecule. In some embodiments, the fusion proteins of the invention include a protein that interacts with Stub1, e.g., E1 or E2, fused to an effector molecule. Exemplary effector molecules include, for example, nucleases, physical blockers, epigenetic recruiters, e.g., a transcriptional repressor, and epigenetic CpG modifiers, e.g., a DNA methylase, a DNA demethylase, a histone modifying agent, or a histone deacetylase, and combinations of any of the foregoing.

In one embodiment, the agents used to decrease Stub1 expression and/or activity are based on CRISPR technology and are RNA-guided nucleases targeting STUB1, or any other protein that interacts with Stub1.

The clustered, regularly interspaced, short palindromic repeat (CRISPR) technology is included in the invention as an approach for generating RNA-guided nuclease with customizable specificities for targeted genome editing. Genome editing mediated by these nucleases has been used to rapidly, easily and efficiently modify endogenous genes in a wide variety of biomedically important cell types and in organisms that have traditionally been challenging to manipulate genetically.

In general, the term “CRISPR system” refers collectively to transcripts and other/elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the invention the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides (e.g., DNA or RNA of STUB1). In some embodiments, a target sequence is located in the nucleus or cytoplasm of a cell.

In preferred embodiments of the invention, the CRISPR/Cas system is a type II CRISPR system and the Cas enzyme is Cas9, which catalyzes DNA cleavage. Enzymatic action by Cas9 derived from Streptococcus pyogenes or any closely related Cas9 generates double-stranded breaks at target site sequences which hybridize to 20 nucleotides of the guide sequence and that have a protospacer-adjacent motif (PAM) sequence NGG following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archae, Mol. Cell 2010, January 15; 37(1): 7.

The type II CRISPR locus from Streptococcus pyogenes SF370 contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. Several aspects of the CRISPR system can be further improved to increase the efficiency and versatility of CRISPR targeting. Optimal Cas9 activity may depend on the availability of free Mg2+ at levels higher than that present in the mammalian nucleus (see e.g., Jinek et al., 2012, Science, 337:816), and the preference for an NGG motif immediately downstream of the protospacer restricts the ability to target on average every 12-bp in the human genome.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence or a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence or tracr sequence are operably linked to and expressed from the same promoter. In addition, electroporation or nucleofection may be used to introduce Cas9 and guide/tracrRNA sequences to cells.

The expression of a target polynucleotide can be modified by allowing a CRISPR complex to bind to the polynucleotide, wherein the CRISPR complex comprises a CRISPR enzyme complexed with a guide sequence hybridized to a target sequence within said polynucleotide, e.g., an RNA-guided nuclease targeting STUB1, wherein said guide sequence is linked to a tracr mate sequence which in turn hybridizes to a tracr sequence. In some embodiments, binding of CRISPR complex to a target polynucleotide results in an increased expression of the target polynucleotide. In another embodiment, binding of CRISPR complex to a target polynucleotide results in a decreased expression of the target polynucleotide (e.g., DNA or RNA of STUB1).

In some embodiments, the fusion protein may comprise an effector, such as a nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

In one embodiment, a fusion protein of the invention may comprise an effector molecule comprising, for example, a CRISPR associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide.

Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzymatic domains, e.g., a dCas9 and a methylase or demethylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional enhancer; a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the site-specific target sequence comprises a sequence within the promoter, enhancer, and/or intronic regions of the target gene.

In some embodiments, the enzyme domain is a Cas9 or a dCas9. In some embodiments, the protein comprises two enzyme domains, e.g., a dCas9 and a transcriptional enhancer or transcriptional repressor domain.

As used herein, a “biologically active portion of an effector domain” is a portion that maintains the function (e.g., completely, partially, minimally) of an effector domain (e.g., a “minimal” or “core” domain).

The chimeric proteins described herein may also comprise a linker, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cas6, Cas7, Cas8, Cas9, CaslO, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the co-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to FokI nuclease (“dCas9-FokI”) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, MA 02139; addgene.org/crispr). A “double nickase” Cas9 that introduces two separate double-strand breaks, each directed by a separate guide RNA, is described as achieving more accurate genome editing by Ran et al. (2013) Cell, 154: 1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, a VP64 domain, a p300 domain (e.g., p300 core domain), an MeCP2 domain, an MQ1 domain, a DNMT3a-3L domain a TET1 domain, and a TET2 domain, protein synthesis inhibitors, nucleases (e.g., Cpf1, Cas9, zinc finger nuclease), fusions of one or more thereof (e.g., dCas9-DNMT, dCas9-APOBEC, dCas9-UGl, dCas9-VP64, dCas9-p300 core, dCas9-KRAB, dCas9-KRAB-MeCP2, dCas9-MQ1, dCas9-DNMT3a-3L, dCAS9-TET1, dCAS9-TET2, and dCas9-MC/MN).

In some embodiments, a suitable nuclease for use in the agent, compositions, and methods of the invention comprises a transcription activator like effector nucleases (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain. Zinc finger polypeptides may be made by fusing the nonspecific DNA cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere, e.g., a conjunction nucleating molecule sequence to act as a steric blocker. In some embodiments, the gRNA is combined with one or more peptides, e.g., S-adenosyl methionine (SAM), that act as a steric presence. In other embodiments, a physical blocker comprises an enzymatically inactive Cas9 polypeptide, or fragment thereof (e.g., dCas9).

In one embodiment, an epigenetic recruiter activates or enhances transcription of a target gene, e.g., a gene that decreases the expression and/or activity of Stub1.

In one embodiment, an epigenetic recruiter silences or represses transcription of a target gene, e.g., a gene that activates the expression and/or activity of Stub1.

In one embodiment, an epigenetic CpG modifier methylates DNA and inactivates or represses transcription.

In one embodiment, an epigenetic CpG modifier demethylates DNA and activates or stimulates transcription.

Antibodies or Antigen Binding Fragments Thereof The agents used in the methods of the present invention further contemplate anti-Stub1 antibodies or antigen binding fragments thereof, thereby decreasing the expression and/or activity of Stub1 in a cell, e.g., an immune cell, and increasing immune response against tumor cell. In one embodiment, the anti-Stub1 antibody, or antigen binding fragment thereof, decreases STUB1 mRNA expression and/or Stub1 protein expression. In another embodiment, the ani-Stub1 antibody, or antigen binding fragment thereof, decreases the activity of Stub1.

In another aspect, the invention also contemplates methods and compositions comprising an antibody which binds to a protein that interacts with Stub1, thereby decreasing the expression and/or activity of the interacting protein, in a cell, and increasing immune response against tumor cells.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from N terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., Stub1). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3rd ed. 1993); (iv) a Fd fragment consisting of the VH and CH1 domains; (v) a Fv fragment consisting of the VL and VH domains of a single arm of an antibody, (vi) a dAb fragment (Ward et al. (1989) Nature 341: 544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “isolated antibody”, as used herein, is intended to refer to an antibody that is substantially free of other antibodies having different antigenic specificities (e.g., an isolated antibody that specifically binds to, e.g., Stub1, is substantially free of antibodies that specifically bind antigens other than Stub1). Moreover, an isolated antibody may be substantially free of other cellular material and/or chemicals. An “isolated antibody” may, however, include polyclonal antibodies, which all bind specifically to, e.g., Stub1.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies are produced by a hybridoma, which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, aptamers, Adnectins (i.e., fibronectin based binding molecules), Affibodies, DARPins, Anticalins, Avimers, and Versabodies all of which employ binding structures that, while they mimic traditional antibody binding, are generated from and function via distinct mechanisms. The embodiments of the instant invention, as they are directed to antibodies, or antigen-binding portions thereof, also apply to the antibody mimetics described above.

As used herein, an antibody that “specifically binds” to an antigen, e.g., Stub1, is intended to refer to an antibody that binds to the antigen with a K_{D} of 1×10⁻⁷ M or less, a K_{D} of 5×10⁻⁸ M or less, a K_{D} of 1×10⁻⁸ M or less, or a K_{D} of 5×10⁻⁹ M or less.

The term “does not substantially bind” to a protein or cells, as used herein, means does not bind or does not bind with a high affinity to the protein or cells, i.e., binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity”, when referring an IgG type antibody, refers to an antibody having a K_{D} of 10⁻⁸ M or less, more preferably 10⁻⁹ M or less and even more preferably 10⁻¹⁰ M or less for, e.g., Stub1. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

Preferably, the antibody binds to Stub1 with a K_{D} of 5×10⁻⁸ M or less, a K_{D} of 1×10⁻⁸ M or less, a K_{D} of 5×10⁻⁹ M or less, or a K_{D} of between 1×10⁻⁸M and 1×10⁻¹⁰ M or less. Standard assays to evaluate the binding ability of the antibodies toward Stub1 are known in the art, including for example, ELISAs, Western blots and RIAs. The binding kinetics (e.g., binding affinity) of the antibodies also can be assessed by standard assays known in the art, such as by ELISA, Scatchard and Biacore analysis.

### Engineered and Modified Antibodies

The V_{H} and/or V_{L} sequences of an antibody prepared according to the methods of the present invention and may be used as starting material to engineer a modified antibody, which modified antibody may have altered properties from the starting antibody.

In some embodiments, the antibody or antibody fragment thereof can be engineered in order to facilitate intracellular delivery of the same. In some embodiments, the antibody or antibody fragment thereof can be fused to a peptide, e.g., a cell penetrating peptide, or a protein transduction domain. In some embodiments, the protein transduction domain or cell penetrating peptides (CPP) comprise 10⁻³⁰ amino acids, primarily based on cationic lysines and arginines and/or hydrophobic amino acids. These peptides translocate across the cell membrane via different mechanisms (Zorko M; Adv. Drug Deliv. Rev 2005, 57 (4), 529-545; Guo Z; Biomed. Reports 2016, 4 (5), 528-534). CPPs have been shown to navigate the membrane in both endocytotic and non-endocytotic pathways (direct cell membrane penetration) depending upon the CPP-cargo combination, the concentration of the cargo and their molecular weights. In some embodiments, nanoparticles and/or liposomes can also be used for intracellular delivery of antibodies.

An antibody can also be engineered by modifying one or more residues within one or both of the original variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al. (1998) Nature 332: 323-327; Jones et al. (1986) Nature 321: 522-525; Queen et al. (1989) Proc. Natl. Acad. Sci. U.S.A. 86: 10029-10033; U.S. Pat. No. 5,225,539 to Winter, and U.S. Pat. Nos. 5,530,101; 5,585,089; 5,693,762 and 6,180,370 to Queen et al.)

Framework sequences for antibodies can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson et al. (1992) J Mol. Biol. 227: 776-798; and Cox et al. (1994) Eur J. Immunol. 24: 827-836; the contents of each of which are expressly incorporated herein by reference. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al. (1997) Nucleic Acids Res. 25: 3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (vbase.mrc-cpe.cam.ac.uk vbase1/list2.php) are translated and the region between and including FR1 through FR3 framework region is retained. The database sequences have an average length of 98 residues. Duplicate sequences, which are exact matches over the entire length of the protein, are removed. A BLAST search for proteins using the program blastp with default, standard parameters except the low complexity filter, which is turned off, and the substitution matrix of BLOSUM62, filters for the top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Identified V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen-binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays known in the art. For example, an antibody of the present invention may be mutated to create a library, which may then be screened for binding to an antigen, e.g., Stub1. Preferably conservative modifications (as discussed above) are introduced. The mutations may be amino acid substitutions, additions or deletions, but are preferably substitutions. Moreover, typically no more than one, two, three, four or five residues within a CDR region are altered.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition, or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. These strategies will be effective as long as the binding of the antibody to an antigen, e.g., Stub1, is not compromised.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields et al. (2001) J. Biol. Chem. 276: 6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C terminus of a full-length heavy chain sequence, as well as the introduction of a cysteine-containing extension to the C terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provides a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol. Bioeng. 87: 614-622). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al. (2002) J Biol. Chem. 277: 26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180). Alternatively, the fucose residues of the antibody may be cleaved off using a fucosidase enzyme. For example, the fucosidase alpha-L-fucosidase removes fucosyl residues from antibodies (Tarentino et al. (1975) Biochem. 14: 5516-5523).

Additionally or alternatively, an antibody can be made that has an altered type of glycosylation, wherein that alteration relates to the level of sialyation of the antibody. Such alterations are described in PCT Publication No. WO/2007/084926 to Dickey et al., and PCT Publication No. WO/2007/055916 to Ravetch et al., both of which are incorporated by reference in their entirety. For example, one may employ an enzymatic reaction with sialidase, such as, for example, Arthrobacter aureofaciens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al. (1975) J. Virol. 15, 882-893 and in Leibiger et al. (1999) Biochem. J. 338, 529-538, respectively. Desialylated antibodies may be further purified by using affinity chromatography. Alternatively, one may employ methods to increase the level of sialyation, such as by employing sialytransferase enzymes. Conditions of such a reaction are generally described in Basset et al. (2000) Scand. J. Immunol. 51: 307-311.

Another modification of the antibodies herein that is contemplated by the invention is pegylation. An antibody can be pegylated to, for example, increase the biological (e.g., serum) half-life of the antibody. To pegylate an antibody, the antibody, or antigen-binding fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al. As such, the methods of pegylation described here also apply to the peptidic molecules of the invention described below.

### Production of Antibodies of the Invention

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also are commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., Stub1. Animals often used for production of polyclonal antibodies are chickens, goats, guinea pigs, hamsters, horses, mice, rats, sheep, and, most commonly, rabbits. Standard methods to produce polyclonal antibodies are widely known in the art and can be combined with the methods of the present invention (e.g., U.S. Pat. Nos. 4,719,290, 6,335,163, 5,789,208, 2,520,076, 2,543,215, and 3,597,409, the entire contents of which are incorporated herein by reference.

Monoclonal antibodies of the present invention can be produced by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g., bacteria) or eukaryotic cells (e.g., yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′).sub.2” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V.sub.H::V.sub.L heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein biomarkers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Following screening and sequencing, antibodies may be produced using recombinant methods and compositions, e.g., as described in U.S. Pat. No. 4,816,567, incorporated by reference herein. An isolated nucleic acid encoding, for example, an anti-Stub1 antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell).

For recombinant production of an anti-Stub1 antibody, a nucleic acid encoding an antibody is isolated and inserted into one or more vectors for further cloning and/or expression in a host cell, Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed, For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell lysate in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat, Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells, Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful, Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MM T 060562); TR 1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa., N.J.), pp. 255-268 (2003).

Antibodies, or antigen binding fragments thereof, described herein are capable of binding to target proteins, such as Stub1, thereby decreasing expression and/or activity of Stub1, and increasing immune responses against tumor cells. In some instances, antibodies, or antigen binding fragments thereof, described herein can decrease the expression and/or activity of Stub1 by at least 20%, e.g., 30%, 40%, 50%, 60%, 70%, 80%, 90%, 95%, or higher. In some instances, antibodies described herein can increase immune response against tumor cells by at least 20%, e.g., 30%, 40%, 50%, 60%, 70%, 80%, 90%, 95%, or higher.

### Modified Immune Cells

In another aspect of the invention, the agents that decrease the expression and/or activity of Stub1 comprise modified immune cells. In some embodiments, the immune cells, e.g., T cells, are derived from a subject and are modified, wherein the expression of an endogenous gene, e.g., STUB1, has been downregulated.

In some embodiments, the modified immune cell comprises a STUB1 knockout T cell. In some embodiments, the STUB1 knockout T cell is postive for IFNγ.

In some embodiments, the modified immune cells, e.g., the immune cells derived from a subject modified to have a decreased level of expression and/or activity of Stub1, are further modified to express a modified T cell receptor, or a chimeric antigen receptor.

In embodiments, prior to expansion and genetic modification or other modifications, a source of immune cells, e.g., T cells, natural killer (NK) cells, or hematopoietic cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain embodiments, immune cells, e.g., T cells, can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using centrifugation. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media. In one aspect, T cells are isolated from peripheral blood lymphocytes by lysing the red blood cells and depleting the monocytes, for example, by centrifugation through a PERCOLL™ gradient or by counterflow centrifugal elutriation.

In some embodiments, the immune cells have been modified wherein the expression of an endogenous gene, e.g., STUB1, has been downregulated. Downregulating expression of an endogenous gene that is involved in suppression of the immune responses to cancer, e.g., STUB1, in immune cells enhances anti-tumor efficacy of the modified immune cells when exposed to an immunosuppressive microenvironment.

In some embodiments, a nucleic acid capable of downregulating endogenous gene expression is introduced, such as by electroporation, transfection, or lenti- or other viral transduction, into the immune cell, e.g., T cell. In another aspect, the invention includes a modified immune cell comprising an electroporated nucleic acid capable of downregulating STUB1 gene expression. In another aspect, the invention includes a pharmaceutical composition comprising the modified immune cell or a modified immune cell generated according to the method described herein and a pharmaceutically acceptable carrier.

In one embodiment, the nucleic acid capable of downregulating endogenous gene expression is selected from the group consisting of an antisense RNA, antigomer RNA, siRNA, shRNA, and a CRISPR system. Endogenous gene expression may be downregulated, knocked-down, decreased, and/or inhibited by, for example, an antisense RNA, antigomer RNA, siRNA, shRNA, a CRISPR system, etc.

As described above, the CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas system can thereby be engineered to cleave virtually any DNA sequence by redesigning the gRNA in cell lines (such as 293T cells), primary cells, and CAR T cells. The CRISPR/Cas system can simultaneously target multiple genomic loci by co-expressing a single Cas9 protein with two or more gRNAs, making this system uniquely suited for multiple gene editing or synergistic activation of target genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, a modified immune cell, e.g., T cell, is generated by introducing a Cas expression vector and a guide nucleic acid sequence specific for a gene into the immune cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

In one embodiment, inducing the Cas expression vector comprises exposing the immune cell to an agent that activates an inducible promoter in the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

The guide nucleic acid sequence is specific for a gene, e.g., STUB1, and targets that gene for Cas endonuclease-induced double strand breaks. The sequence of the guide nucleic acid sequence may be within a loci of the gene, or outside the gene loci, e.g., within an enhancer region. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In some embodiments, the modified immune cells are further modified to express a modified T cell receptor, or a chimeric antigen receptor, wherein the modified immune cells exhibit an antitumor property.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting the specificity of a T cell expressing the CAR specific or tumor associated antigens.

### CAR T Cells

The modified immune cells of the present invention also include modified chimeric antigen receptor (CAR) T cells. CAR T cell therapy has been revolutionary as it has produced remarkably effective and durable clinical responses for cancer treatment. In this strategy, a patient's own T cells are genetically engineered to express a synthetic receptor that binds a tumor antigen. CAR T cells are then expanded for clinical use and infused back into the patient's body to attack and destroy chemotherapy-resistant cancer. Dramatic clinical responses and high rates of complete remission have been observed in the setting of CAR T-cell therapy of B-cell malignancies. This resulted in recent FDA approvals of CAR T cells directed against the CD19 protein for treatment of acute lymphoblastic leukemia and diffuse large B-cell lymphoma and FDA approvals of CAR T cells for treating multiple myeloma, as well as ongoing development of CAR T cells for treating non-B cell hematologic malignancy. Thus, CAR T cells represent one of the first successful examples of synthetic biology and personalized cellular cancer therapy to become commercially available.

The modified immune cells of the present invention include modified CAR T cells having a decreased level of expression and/or activity of Stub1. In some embodiments, the modified immune cell comprises a STUB1 knockout (KO) CAR T cell, wherein the CAR includes an antigen binding domain, a transmembrane domain and an intracellular domain of a co-stimulatory molecule. In some embodiments, the STUB1 knockout CAR T cell is postive for IFNγ.

One or more domains or a fragment of a domain of the CAR may be human. In one embodiment, the present invention includes a fully human CAR. The nucleic acid sequences coding for the desired domains can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than as a cloned molecule.

In one embodiment, the CAR comprises an antigen binding domain that binds to an antigen on a target cell. Examples of cell surface markers that may act as an antigen that binds to the antigen binding domain of the CAR include those associated with viral, bacterial and parasitic infections, autoimmune disease, and cancer cells.

The choice of antigen binding domain depends upon the type and number of antigens that are present on the surface of a target cell. For example, the antigen binding domain may be chosen to recognize an antigen that acts as a cell surface marker on a target cell associated with a particular disease state.

In one embodiment, the antigen binding domain binds to a tumor antigen, such as an antigen that is specific for a tumor or cancer of interest. In one embodiment, the tumor antigen of the present invention comprises one or more antigenic cancer epitopes.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof.

The antigen binding domain may bind one or more antigens, such as but not limited to CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5.Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp 100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART 1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some instances, it is beneficial for the antigen binding domain to be derived from the same species in which the CAR will ultimately be used in. For example, for use in humans, it may be beneficial for the antigen binding domain of the CAR to comprise a human antibody, humanized antibody as described elsewhere herein, or a fragment thereof.

It is also beneficial that the antigen binding domain is operably linked to another domain of the CAR, such as the transmembrane domain or the intracellular domain, both described elsewhere herein, for expression in the cell. In one embodiment, a nucleic acid encoding the antigen binding domain is operably linked to a nucleic acid encoding a transmembrane domain and a nucleic acid encoding an intracellular domain.

With respect to the transmembrane domain, the CAR can be designed to comprise a transmembrane domain that connects the antigen binding domain of the CAR to the intracellular domain. In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines.

In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use in the invention include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability. In one embodiment, the intracellular domain of the CAR comprises dual signaling domains. The dual signaling domains may include a fragment or domain from any of the molecules described herein.

Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In one embodiment, the intracellular domain of the CAR includes any portion of a co-stimulatory molecule, such as at least one signaling domain from CD3, CD27, CD28, ICOS, 4-1BB, OX40, CD226, T cell receptor (TCR), any derivative or variant thereof, any synthetic sequence thereof that has the same functional capability, and any combination thereof.

Between the antigen binding domain and the transmembrane domain of the CAR, or between the intracellular domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between the transmembrane domain and the intracellular domain of the CAR. An example of a linker includes a glycine-serine doublet.

In some embodiments, the cells are transformed with the CAR and the CAR is expressed on the cell surface. In some embodiments, the cells are transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell is transfected with a nucleic acid, e.g., mRNA, cDNA, DNA, encoding a CAR. In some such embodiments, the cell may transiently express the CAR. Furthermore, the present invention provides CAR-expressing cell, e.g., CAR-T compositions and their use in medicaments or methods for treating cancer.

In one embodiment, the modified T cell further comprises an exogenous nucleic acid encoding a modified TCR comprising affinity for a surface antigen on a target cell. The invention also includes a population of cells comprising the modified T cell described herein.

### Small Molecules

In another aspect of the invention, the agent that decreases the expression and/or activity of Stub1 in an immune cell is a small molecule.

The small molecules of the instant invention are characterized by particular functional features or properties. For example, the small molecules bind to Stub1, or any other protein that interacts with Stub1, thereby decreasing Stub1 activity in an immune cell, e.g., T cells.

The terms “small molecule compounds”, “small molecule drugs”, “small molecules”, or “small molecule inhibitors” are used interchangeably herein to refer to the compounds of the present invention screened for an effect on Stub1 and their ability to decrease the activity of Stub1. These compounds may comprise compounds in PubChem database (pubchem.ncbi.nlm.nih.gov/), the Molecular Libraries Screening Center Network (MLSCN) database, compounds in related databases, or derivatives and/or functional analogues thereof.

As used herein, “analogue” or “functional analogue” refers to a chemical compound or small molecule inhibitor that is structurally similar to a parent compound, but differs slightly in composition (e.g., one or more atoms or functional groups are added, removed, or modified). The analogue may or may not have different chemical or physical properties than the original compound and may or may not have improved biological and/or chemical activity. For example, the analogue may be more hydrophobic or it may have altered activity (increased, decreased, or identical to parent compound) as compared to the parent compound. The analogue may be a naturally or non-naturally occurring (e.g., recombinant) variant of the original compound. Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound or small molecule inhibitor that is structurally similar to a parent compound and (actually or theoretically) derivable from that parent compound. A “derivative” differs from an “analogue” or “functional analogue” in that a parent compound may be the starting material to generate a “derivative,” whereas the parent compound may not necessarily be used as the starting material to generate an “analogue” or “functional analogue.” A derivative may or may not have different chemical or physical properties of the parent compound. For example, the derivative may be more hydrophilic or it may have altered reactivity as compared to the parent compound. Derivatization (i.e., modification by chemical or other means) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al. (1996) Adv. Drug Deliv. Rev. 19: 115; Design of Prodrugs, H. Bundgaard (ed.), Elsevier, 1985; and H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts, calcium salts, and salts with physiologically tolerable quaternary ammonium ions and acid addition salts with ammonia and physiologically tolerable organic amines such as triethylamine, ethanolamine, or tris-(2-hydroxyethyl)amine). Basic groups can form acid addition salts, for example with inorganic acids such as hydrochloric acid (“HCl”), sulfuric acid, or phosphoric acid, or with organic carboxylic acids and sulfonic acids such as acetic acid, citric acid, benzoic acid, maleic acid, fumaric acid, tartaric acid, methanesulfonic acid, or p-toluenesulfonic acid. Compounds that simultaneously contain a basic group and an acidic group such as a carboxyl group in addition to basic nitrogen atoms can be present as zwitterions. Salts can be obtained by customary methods known to those skilled in the art, for example, by combining a compound with an inorganic or organic acid or base in a solvent or diluent, or from other salts by cation exchange or anion exchange.

Small molecules are known to have molecular weights of 1200 Daltons or below, 1000 Daltons or below, 900 Daltons or below, 800 Daltons or below, 700 Daltons or below, 600 Daltons or below, 500 Daltons or below, 400 Daltons or below, 300 Daltons or below, 200 Daltons or below, 100 Daltons or below, 50 Daltons or below, 25 Daltons or below, or 10 Daltons or below.

The small molecules of the present invention are selected or designed to bind to Stub1, or any other protein that interacts with Stub1.

In preferred embodiments, a small molecule of the invention binds to Stub1, or any other protein that interacts with Stub1 with high affinity, for example, with an affinity of a K_{D} of 1×10⁻⁷ M or less, a K_{D} of 5×10⁻⁸ M or less, a K_{D} of 1×10⁻⁸ M or less, a K_{D} of 5×10⁻⁹ M or less, or a K_{D} of between 1×10⁻⁸ M and 1×10⁻¹⁰ M or less.

Small molecules of the invention may be made or selected by several methods known in the art and by methods as described herein. Screening procedures can be used to identify small molecules from libraries which bind Stub1, or any other protein that interacts with Stub1.

### Peptidic Molecules

In another aspect of the invention, the agent that decreases the expression and/or activity of Stub1 in an immune cell is a peptidic molecule.

In one embodiment, the peptidic moieties of the invention may comprise an entire protein domain of Stub1. In some embodiments, the peptidic moieties of the invention may have as little as 50% identity to Stub1, e.g., a peptidic moiety of the invention may be at least 50% identical, at least 60% identical, at least 70% identical, at least 80% identical, at least 90% identical, or at least 95%, 96%, 97%, or 98% identical to Stub1.

In another embodiment, the peptidic moieties of the invention may comprise an entire protein domain of any other protein that interacts with Stub1. In some embodiments, the peptidic moieties of the invention may have as little as 50% identity to the protein that interacts with Stub1, for example, a peptidic moiety of the invention may be at least 50% identical, at least 60% identical, at least 70% identical, at least 80% identical, at least 90% identical, or at least 95%, 96%, 97%, or 98% identical to the protein that interacts with Stub1.

A peptidic moiety of the invention may bind to contiguous or non-contiguous amino acid residues of Stub1, or any other protein that interacts with Stub1.

A peptide molecule of the invention may be further modified to increase its stability, bioavailability or solubility. For example, the peptide molecule, e.g., the entire protein of Stub1, or a fragment of Stub1, may be fused to a degron tag. As used herein the term “degron tag” refers to a portion of a protein that is important in regulation of protein degradation rates. Degron tags allow the precise and well-controlled analysis of essential genes by rapidly inducing degradation of the protein of interest.

In some embodiments, one or more L-amino acid residues within the peptidic molecules may be replaced with a D-amino acid residue. The term “mimetic” as applied to the peptidic molecules of the present invention is intended to include molecules that mimic the chemical structure of a D-peptidic structure and retain the functional properties of the D-peptidic structure. The term “mimetic” is further intended to encompass an “analogue” and/or “derivative” of a peptide as described below. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball and Alewood (1990) J. Mol. Recognition 3: 55; Morgan and Gainor (1989) Ann. Rep. Med. Chem. 24: 243; and Freidinger (1989) Trends Pharmacol. Sci. 10: 270. See also Sawyer (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith et al. (1995) J. Am. Chem. Soc. 117: 11113-11123; Smith et al. (1994) J. Am. Chem. Soc. 116: 9947-9962; and Hirschman et al. (1993) J. Am. Chem. Soc. 115: 12550-12568.

Other methods to stabilize peptides and peptide structures may be used, e.g., olefinic cross-linking of helices through O-allyl serine residues (Blackwell, H. E.; Grubbs, R. H. Angew. Chem., Int. Ed. 1998, 37, 3281-3284, incorporated herein by reference), all-hydrocarbon cross-linking (Schafmeister and Verdine J. Am. Chem. Soc. 2000, 122 (24), 5891-5892, incorporated herein by reference) and the methods disclosed in U.S. Pat. No. 7,183,059 (incorporated herein by reference). The methods disclosed in Blackwell et al. and Schafmeister et al. may be described as producing “stapled” peptides, i.e., peptides which are covalently locked into a particular conformational state or secondary structure, or peptides which have a particular intramolecular covalent linkage which predisposes them to form a particular conformation or structure. If a peptide thus treated is predisposed to, e.g., form an alpha-helix which is important for target binding, then the energetic threshold for binding will be lowered. Such “stapled” peptides have been shown to be resistant to proteases and may also be designed to cross the cellular membrane more effectively (also see Walensky et al. Science 2004:Vol. 305. no. 5689, pp. 1466-1470; Bernal et al. J Am Chem Soc. 2007, 129(9):2456-7 which are incorporated herein by reference). Accordingly, peptides of the invention may be thus stapled or otherwise modified to lock them into a specific conformational shape or they may be modified to be predisposed to particular conformation or secondary structure which is beneficial for binding. It is contemplated that such peptide modifications may occur prior to peptide selection such that the benefit of any conformational constraints may also be selected for. Alternatively, in some embodiments, the modifications may be made after selection to preserve a conformation known to be beneficial to binding or to further enhance a peptide candidate. See also, WO/2010/033617, the entire contents of which are incorporated herein by reference.

Other methods to stabilize peptides and peptide structures include linking the amino and carboxy termini of a protein with a peptide bond to form a circular or cyclic peptide. See, e.g., WO/2008/07489 and U.S. Pat. No. 55,726,287, the entire contents of each of which are incorporated herein by reference.

As used herein, a “derivative” of a peptidic molecule of the invention refers to a form of the peptidic molecule in which one or more reaction groups on the molecule have been derivatized with a substituent group. Examples of peptide derivatives include peptides in which an amino acid side chain, the peptide backbone, or the N or C terminus has been derivatized (e.g., peptidic compounds with methylated amide linkages). As used herein an “analogue” of a peptidic molecule of the invention refers to a peptidic molecule that retains chemical structures of the molecule necessary for functional activity of the molecule yet also contains certain chemical structures that differ from the molecule. An example of an analogue of a naturally-occurring peptide is a peptide that includes one or more non-naturally-occurring amino acids. As used herein, a “mimetic” of a peptidic molecule of the invention refers to a peptidic molecule in which chemical structures of the molecule necessary for functional activity of the molecule have been replaced with other chemical structures that mimic the conformation of the molecule. Examples of peptidomimetics include peptidic compounds in which the peptide backbone is substituted with one or more benzodiazepine molecules (see e.g., James et al. (1993) Science 260: 1937-1942).

Analogues of the peptidic molecules of the invention are intended to include molecules in which one or more L- or D-amino acids of the peptidic structure are substituted with a homologous amino acid such that the properties of the molecule are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-limiting examples of homologous substitutions that can be made in the structures of the peptidic molecules of the invention include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain.

The term “mimetic,” and in particular, “peptidomimetic,” is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets.

Other possible modifications include an N-alkyl (or aryl) substitution (ψ[CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

Peptidic molecules of the present invention may be made by standard methods known in the art. The peptidic molecule may be cloned from human cells using standard techniques, inserted into a recombinant vector, and expressed in an in vitro cell system (e.g., by transfection of the vector into yeast cells). Alternatively, the peptidic molecules may be designed and synthesized de novo via known synthesis methods such as Atherton et al. (1989) Oxford, England: IRL Press. ISBN 0199630674; Stewart et al. (1984) 2nd edition, Rockford: Pierce Chemical Company, 91. ISBN 0935940030; Merrifield (1963) J Am. Chem. Soc. 85: 2149-2154.

The peptidic molecules can then be tested for functional activity using any of the assays described herein, e.g., those described in the Examples section below.

### Screening Methods

In certain aspects, the present application provides a method for identifying a compound that modulates, e.g., increase or decrease, the expression and/or activity of Stub1. Compounds that are capable of decreasing the expression, stability, and/or activity of Stub1 or its binding partners, as identified by the methods of the invention, are useful as candidates to treat cancer, reduce tumor size, or prolong the survival of a subject in need thereof, or to increase an immune response against a tumor in a subject in need thereof.

For example, in one aspect, the present invention provides methods for identifying a compound useful for treating a cancer in a subject. The methods include providing a test compound (or a plurality of test compounds), determining the effect of the test compound on the expression and/or activity of Stub1, and selecting a compound which modulates the expression and/or activity of Stub1, thereby identifying a compound useful for treating a cancer in the subject. In some embodiments, a decrease in the expression and/or activity of Stub1 indicates that the compound is useful for treating a cancer.

In another aspect, the present invention provides methods for identifying a compound useful for increasing an immune response against a tumor in a subject in need thereof. The methods include providing a test compound (or a plurality of test compounds), determining the effect of the test compound on the expression and/or activity of Stub1, and selecting a compound which decreases the expression and/or activity of Stub1, thereby identifying a compound useful for increasing an immune response against the tumor in the subject. In another aspect, DNA-encoded library screens may be used to identify compounds that bind and may inhibit Stub1.

Examples of modulators, candidate compounds or test compounds include, but are not limited to, nucleic acids (e.g., DNA and RNA), carbohydrates, lipids, proteins, peptides, peptidomimetics, small molecules and other drugs. Modulators can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. Nos. 5,738,996; and 5,807,683, the entire contents of each of the foregoing references are incorporated herein by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, the entire contents of each of the foregoing references are incorporated herein by reference. Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). The entire contents of each of the foregoing references are incorporated herein by reference.

The test compound can be contacted with a cell that expresses the Stub1 protein or a molecule with which Stub1 directly interacts. For example, the test compound can be contacted with a cell that naturally expresses or has been engineered to express the protein(s) by introducing into the cell an expression vector encoding the protein.

Alternatively, the test compounds can be subjected to a cell-free composition that includes the protein(s) (e.g., a cell extract or a composition that includes e.g., purified natural or recombinant protein).

Compounds that modulate expression and/or activity of Stub1, or a binding partner of Stub1 can be identified using various “read-outs.” For example, a cell can be transfected with an expression vector, incubated in the presence and in the absence of a test compound, and the effect of the compound on the expression of Stub1 or on a biological response regulated by Stub1 can be determined. The biological activities of Stub1 include activities determined in vivo, or in vitro, according to standard techniques. Activity can be a direct activity, such as an association with a binding partner, or ubiquitination. Alternatively, the activity is an indirect activity, such as an increase in immune response.

To determine whether a test compound modulates Stub1 protein expression, proteomics or protein quantification assays can be performed. To determine whether a test compound modulates STUB1 mRNA expression, various methodologies can be performed, such as quantitative or real-time PCR.

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein, or luciferase. Standard methods for measuring the activity of these gene products are known in the art.

A variety of cell types are suitable for use as an indicator cell in the screening assay. Preferably a cell line or a primary cell is used which expresses low levels of endogenous Stub1 and is then engineered to express recombinant protein. Cells for use in the subject assays include eukaryotic cells. For example, in one embodiment, a cell is a fungal cell, such as a yeast cell. In another embodiment, a cell is a plant cell. In yet another embodiment, a cell is a vertebrate cell, e.g., an avian cell or a mammalian cell (e.g., a murine cell, or a human cell).

Recombinant expression vectors that can be used for expression of, e.g., Stub1, are known in the art. For example, the cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for, or a molecule in a signal transduction pathway involving (e.g., human, murine and yeast), are known in the art and can be used for the design of PCR primers that allow for amplification of a cDNA by standard PCR methods or for the design of a hybridization probe that can be used to screen a cDNA library using standard hybridization methods.

In another embodiment, the test compounds can be subjected to a cell-free composition that includes the protein(s) (e.g., a cell extract or a composition that includes e.g., either purified natural or recombinant protein). Stub1 expressed by recombinant methods in host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate Stub1 activity or the activity of a binding partner in a signal transduction pathway involving Stub1 are identified based on their ability to modulate the interaction of Stub1 with its binding partner. In another embodiment, compounds that specifically modulate Stub1 activity or the activity of a binding partner in a signal transduction pathway involving Stub1 are identified based on their ability to modulate the post-translation modification of Stub1 and its binding partner. The binding partner can be a mRNA molecule or a protein molecule. Suitable assays are known in the art that allow for the detection of protein-protein interactions (e.g., immunoprecipitations, two-hybrid assays and the like) or that allow for the detection of interactions between Stub1 and an mRNA (e.g., electrophoretic mobility shift assays, DNAse I footprinting assays and the like). By performing such assays in the presence and absence of test compounds, these assays can be used to identify compounds that modulate (e.g., decrease or enhance) the activity of Stub1 with a binding partner.

Compounds identified in the subject screening assays can be used in methods of modulating one or more of the biological responses regulated by Stub1. It will be understood that it may be desirable to formulate such compound(s) as pharmaceutical compositions as described herein prior to contacting them with cells.

Once a test compound is identified that directly or indirectly modulates, e.g., Stub1 expression or activity by one of the variety of methods described hereinbefore, the selected test compound (or “compound of interest”) can then be further evaluated for its effect on cells, for example by contacting the compound of interest with cells either in vivo (e.g., by administering the compound of interest to an organism) or ex vivo (e.g., by isolating cells from an organism and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

In another aspect, the invention pertains to a combination of two or more of the assays described herein. For example, a modulator can be identified using a cell-based or a cell-free assay, and the ability of the modulators to increase or decrease the activity of Stub1 or a protein with which Stub1 interacts can be confirmed in vivo, e.g., in an animal, such as, for example, an animal model for, e.g., a tumor model.

Moreover, a modulator of Stub1 or a molecule in a signaling pathway involving Stub1 identified as described herein (e.g., an antisense nucleic acid molecule, or a specific antibody, or a small molecule) can be used in an animal model to determine the efficacy, toxicity, or side effects of treatment with such a modulator. In some embodiments, a modulator identified as described herein can be used to reduce the tumor size, increase the survival (e.g., overall or progression-free) and/or improve immune response of an animal model. Alternatively, a modulator identified as described herein can be used in an animal model to determine the mechanism of action of such a modulator.

In another embodiment, it will be understood that similar screening assays can be used to identify compounds that indirectly modulate the activity and/or expression of Stub1, e.g., by performing screening assays such as those described above using molecules with which Stub1 interacts, or any molecules that act either upstream or downstream of Stub1 in the pathway.

Compounds identified by the screening assays of the present invention are considered as candidate therapeutic compounds useful for treating diseases, e.g., cancer, or autoimmune disease, as described herein. Thus, the invention also includes compounds identified in the screening assays, and methods for their administration and use in the treatment, prevention, or delay of development or progression of diseases described herein.

### IV. Pharmaceutical Compositions

Agents that decrease the expression and/or the activity of Stub1, e.g., inhibitory nucleic acids, small molecule inhibitors, modified immune cells, peptidic molecules, and/or anti-Stub1 antibodies, or antigen binding fragments thereof, as described herein, may be formulated into pharmaceutical compositions suitable for administration in human or non-human subjects. Such pharmaceutical compositions may be intended for therapeutic use, or prophylactic use. One or more of the agents can be mixed with a pharmaceutically acceptable carrier (excipient), including buffer, to form a pharmaceutical composition for administering to a patient who may benefit from reduced Stub1 activity in vivo.

“Pharmaceutically acceptable” means that the carrier must be compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20^{th} Ed. (2000) Lippincott Williams and Wilkins, Ed. K. E. Hoover. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In one example, a pharmaceutical composition described herein contains more than one agent that decreases the expression and/or activity of Stub1.

In some examples, the pharmaceutical composition described herein comprises emulsion-based or lipid-based formulations, such as liposomes containing an agent that decreases the expression and/or activity of Stub1, e.g., a small molecule inhibitor of Stub1, which can be prepared by any suitable method, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The agent that decreases the expression and/or activity of Stub1 may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20^{th} Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, or antigen-binding portion thereof, which matrices are in the form of shaped articles, e.g., films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions may comprise a modified immune cell, e.g., a modified immune cell having a decreased level of expression and/or activity of Stub1, e.g., a CAR T cell, e.g., STUB1 knockout (KO) CAR T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated, e.g., for intravenous administration.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 mg to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Jnfonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an agent that decreases the expression and/or activity of Stub1, e.g., a small molecule inhibitor of Stub1, and/or anti-Stub1 antibody or antigen-binding portion thereof, with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

When “an effective amount” or “therapeutic amount” is indicated, the precise amount of the compositions to be administered can be determined by a physician with consideration of individual differences in age, weight, tumor size, extent of infection or metastasis, and condition of the patient (subject).

In some embodiments, pharmaceutical compositions comprising an agent that decreases the expression and/or activity of Stub1, e.g., a small molecule inhibitor of Stub1, and/or anti-Stub1 antibody or antigen-binding portion thereof, as described herein, may be administered at a weight-based dose, e.g., a dosage of about 0.01-10 ng/kg (e.g., about 0.1-5 mg/kg). For example, the composition is administered at a dosage of about 0.01, 0.02, 0.05, 0.1, 0.3, 0.5, 1, 3, 5, 10, 15, 20, 25, 30, 35, 40, 45, 50 mg/kg. In some embodiments, the composition is administered every day, every other day, every 2 days, every 3 days, every 4 days, every 5 days, or every 6 days. In other embodiments, the composition is administered every 1-10 weeks (e.g., every 1, 2, 3, 4, 5, 6, 7, 8, 9, or 10 weeks). For example, the composition is administered for a total of 7 days to 3 years (e.g., 7 days, 14 days, 3 weeks, 4 weeks, 5 weeks, 6 weeks, 7 weeks, 8 weeks, 9 weeks, 10 weeks, 11 weeks, 12 weeks, 24 weeks, 36 weeks, 1 year, 1.5 years, 2 years, 2.5 years, or 3 years). In some embodiments, the composition is provided in a fix dosage of about 0.01-50 mg (e.g., about 0.05-30 mg) per dose. For example, the composition is administered in a dosage of about 0.01, 0.02, 0.05 mg, 0.1 mg, 0.2 mg, 0.4 mg. 0.8 mg, 2 mg, 5 mg. 10 mg. 15 mg. 20 mg. 25 mg, 30 mg, 40 mg, or 50 mg) per administration (e.g., per injection).

In some embodiments, the pharmaceutical composition comprises a modified immune cell, wherein the modified immune cells comprise immune cells derived from a subject and are modified to have a decreased level of expression and/or activity of Stub 1, e.g. STUB1 knockout CAR T cells. It can generally be stated that a pharmaceutical composition comprising the modified immune cells (e.g., T cells, NK cells) described herein may be administered at a dosage of about 10⁴ to about 10¹¹ cells/kg body weight, in some instances about 10⁵ to about 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. 1. of Med. 319: 1676, 1988).

In some embodiments, a dose of modified immune cells, e.g., CAR T cells, e.g., STUB1 knockout CAR T cells, comprises about 1×10⁶ to about 1×10¹⁰ cells/kg. In some embodiments, a dose of modified immune cells, e.g., CAR T cells, e.g., STUB1 knockout CAR T cells, comprises about 1×10⁶, about 2×10⁶, about 5×10⁶, about 1×10⁷, about 2×10⁷, about 5×10⁷, about 1×10⁸, about 2×10⁸, about 5×10⁸, about 1×10⁹, about 2×10⁹, about 5×10⁹, or about 1×10¹⁰ cells/kg.

The administration of the subject compositions may be carried out in any convenient manner. The compositions described herein may be administered to a patient trans arterially, subcutaneously, intradernmally, intratumorally, intranodally, intramedullary, intramuscularly, intrathecally, by intravenous (i.v.) injection, or intraperitoneally, e.g.. by intradermal or subcutaneous injection. The compositions of immune cells (e._. T cells, NK cells) may be injected directly into a tumor, lymph node, or site of infection.

### V. Kits

The invention also provides compositions and kits for inhibiting, treating or monitoring a disease or disorder associated with Stub1, e.g., cancer, recurrence of a disease or disorder associated with Stub1, e.g., cancer, or survival of a subject being treated for a disease or disorder associated with Stub1, e.g., cancer. These kits may include one or more agents that decrease the expression and/or activity of Stub1 and instructions for use. The kit can further contain one more additional reagent, such as an additional therapeutic agent, e.g., an immunosuppressive reagent, a cytotoxic agent or a radiotoxic agent or one or more additional agents of the invention, as described herein. Kits typically include a label indicating the intended use of the contents of the kit. The term label includes any writing, or recorded material supplied on or with the kit, or which otherwise accompanies the kit.

In certain embodiments, the kits can also comprise, e.g., a buffering agent, a preservative, a protein stabilizing agent, reaction buffers. Each component of the kit can be enclosed within an individual container and all of the various containers can be within a single package, along with instructions for interpreting the results of the assays performed using the kit.

The kits of the invention may optionally comprise additional components useful for performing the methods of the invention.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1. Identification of Stub1 as a Putative Negative Regulator of CD8+ T Cells in an In Vivo Screen

A genetic screen was performed to identify genes that when deleted in CD8+ T cells led to an enrichment of these CD8+ T cells in tumors. The enrichment of CD8+ T cells in which a gene was deleted is consistent with a negative regulatory role for the gene. This screen was performed twice to assess the consistency of the hits.

Briefly, a 2747 gRNA library was created consisting of 899 genes (including the positive control genes Pdcd1 and Ptpn2) at 3 gRNAs/gene as well as 50 negative control gRNAs. This library was cloned into a custom lentiviral plasmid that encodes a gRNA expression cassette and the transduction reporter Thy1.1. Lentivirus was prepared using this plasmid library and 300K naive CD8+ T cells from OT-1+Cas9-expressing female mice were transduced with the library using the transduction enhancers Retronectin and LentiBOOST™. OT-1 is a T cell receptor (TCR) transgenic T cell that recognizes the peptide SIINFEKL (SEQ ID NO:7) that the tumor cells were engineered to express. Cas9 binds to gRNAs following expression from the lentiviral construct to induce KO of target genes. The transduced naive CD8+ T cells were cultured in vitro in RPMI media containing fetal bovine serum, penicillin-streptomycin, HEPES, non-essential amino acids, sodium pyruvate, and murine IL-7 for 7 days. The cultured T cells were then stained for Thy1.1 (to identify transduced cells) and sorted for live Thy1.1+ naive CD8+ T cells. The Thy1.1+ cells were then either (1) frozen for input sequencing of gRNAs (baseline) or (2) injected intravenously into Cas9-expressing Thy1.2+ female mice. Note Cas9-expressing mice are required as recipients because they are tolerized to the Cas9 antigen (and are used to ensure that the transferred Cas9-expressing T cells are not eliminated by the immune system). In addition, Thy1.2+ mice were used to enable identification of transferred Thy1.1+ cells by flow cytometry. The next day, these recipient mice were injected subcutaneously with B16 melanoma tumor cells modified to express SIINFEKL (SEQ ID NO:7) (which is recognized by OT-1+ T cells). Tumor formation and growth were monitored for 13 days. Tumors and tumor-draining lymph nodes (inguinal lymph node on tumor side) were isolated and pooled into sets of 10 to aid in processing. The transferred T cells (Thy1.1-expressing) were sorted from each of these organs using flow cytometry and frozen for output sequencing of gRNAs. The next day, genomic DNA was isolated from the input/output cell pellets and submitted for sequencing. PCR was performed to specifically amplify the gRNAs from the genomic DNA and add sequencing adapters. These PCR products were sequenced on a HiSeq and sequencing data were mapped back to the original gRNA library to obtain a count matrix of gRNAs and their abundancies in each sample. These count tables (raw mapped reads) were then log-normalized to account for slightly different sequencing depths of each sample. Log-normalized read counts for output samples were then baseline-normalized to the input samples to determine the log-normalized fold changes of each gRNA from output to input. These fold changes were then ranked from highest to lowest (most enriched gRNA to most depleted) and a hypergeometric p-value was calculated. Note average −log 10(p-values) of ˜3 are considered highly significant. The fold changes were also normalized to the 50 negative control gRNAs by z-scoring (subtracting the average fold change of negative control RNAs from each gRNA of interest and dividing by the standard deviation of the negative control gRNAs). This converts the data into a more understandable metric of number of standard deviations from the negative controls.

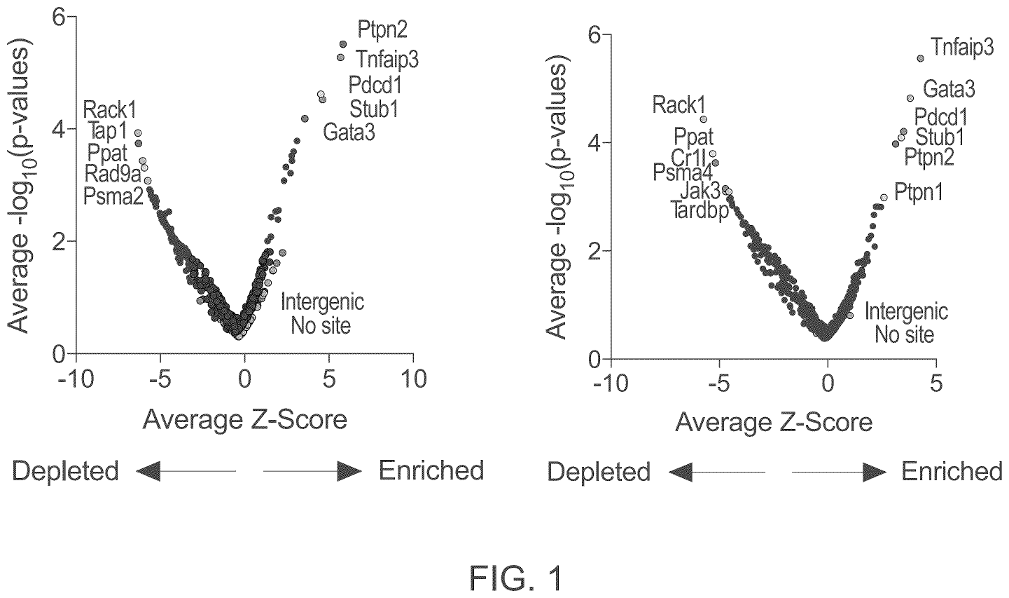

Several known regulators of CD8+ T cell abundance in tumors were identified, e.g., Rack1, Tap1, Ptpn2, Tnfaip3, Pdcd1, and Gata3, and novel regulators, e.g., Stub1 and Ppat, were identified as well (FIG. 1). Importantly, the positive controls for enrichment (Pdcd1 and Ptpn2) were indeed enriched, indicating the screen was of high quality. Stub1 was selected for subsequent studies because of its novelty, enrichment in tumors, and consistency as a top hit in two independent screens.

Thus, Stub1 was identified as a putative negative regulator of CD8+ T cell abundance in tumors.

### Example 2. Validation of In Vivo Screen with a Competitive Assay Showing Stub1 Enhances CD8+ T Cell Function

A competition assay was subsequently performed to validate the in vivo screen results.

The assay compared a control and a KO T cell in the same mouse, enabling comparison of the two T cell types in the same tissue microenvironment. The two populations started at equivalent ratios, and following tumor growth, these populations were evaluated to determine if this ratio had changed. A validated target would result in a competitive advantage of the T cells with gene KO. This experiment was performed twice to ensure reproducibility.

The top scoring gRNA in the screens from Experiment 1 were identified: Ptpn1, Gata3, Stub1, and Tnfaip3. A second independent gRNA was designed and was not present in the screen for each of these genes. An orthogonal approach, called nucleofection, to the lentiviral approach in Experiment 1, was used to knockout these genes as well as control (non-targeting) and Ptpn2 (positive control) genes. Note CD45.1 congenically marked T cells were nucleofected with a control gRNA, and CD45.1 and CD45.2 congenically T cells were nucleofected with control, Ptpn2, Ptpn1, Gata3, Stub1, and Tnfaip3 gRNAs.

Briefly, OT-1+ naive CD8+ T cells congenically marked with CD45.1 or CD45.1 and CD45.2 (CD45.1/.2) were electroporated with Cas9 protein-gRNA ribonucleoprotein complexes to transiently deliver Cas9/gRNA to the nucleus, where they would knockout the gene of interest. The nucleofected naive CD8+ T cells were cultured in vitro in RPMI media containing fetal bovine serum, penicillin-streptomycin, HEPES, non-essential amino acids, sodium pyruvate, and murine IL-7 for 1 day. The next day, cells were counted and mixed in a 50:50 ratio (CD45.1 control gRNA and CD45.1/.2 control, Ptpn1, Ptpn2, Gata3, Stub1, or Tnfaip3 gRNAs). These cells were (1) transferred to CD45.2 wild-type recipients intravenously or (2) stained with CD45.1 and CD45.2 to assess the exact ratio of cells (should be close to 50:50). Five days later, mice were injected subcutaneously with B16 melanoma tumor cells modified to express SIINFEKL (SEQ ID NO:7) (which is recognized by OT-1+ T cells). Tumor formation and growth was monitored for approximately 10 days. Tumors were isolated and congenically marked transferred cells (CD45.1 and CD45.1/.2) assessed by flow cytometry. The ratio of CD45.1 to CD45.1/.2 at output was compared to the input ratio and the log-normalized fold change calculated. All sample ratios were compared to control vs. control for significance.

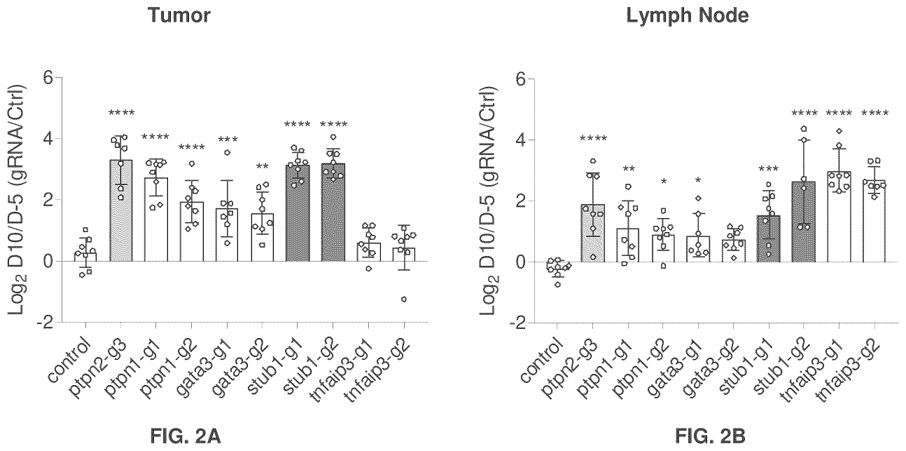

The gRNAs for Ptpn1, Gata3, and Stub1 were significantly enriched in tumor compared to the control gRNA mix (FIG. 2A). The gRNAs for Ptpn1, Gata3, Stub1 and Tnfaip3 were significantly enriched in tumor-draining lymph node (LN) compared to the control gRNA mix (FIG. 2B). In addition, the positive control Ptpn2 was significantly enriched. Notably, Stub1 KO CD8+ T cells were equivalently enriched to Ptpn2 KO CD8+ T cells. Ptpn2 was previously identified as a potent inhibitor of CD8+ T cells. These results indicate that Stub1 deletion strongly enhances CD8+ T cell function and point to a significant inhibitory function for Stub1 in CD8+ T cells.

### Example 3. Assessing Stub1 Knockout (KO) T Cells in an In Vivo Tumor Growth Control Assay Shows Stub1 KO CD8+ T Cells Control Tumors Significantly Better than Control CD8+ T Cells

To determine whether Stub1 KO CD8+ T cells control tumor growth more effectively than control CD8+ T cells, a tumor growth control assay was performed using control and Stub1 KO T cells. For this experiment, control and Stub 1 KO OT-1+ T cell populations were transferred into separate mice. These mice were injected with B16 melanoma tumor cells expressing SIINFEKL (SEQ ID NO:7) (which is recognized by OT-1+ T cells), and tumor growth was monitored. Tumor growth in mice with Stub1 KO OT-1+ T cells was compared with mice receiving the control cells.

Stub1 KO naive OT-1+CD8+ T cells and control cells were created using nucleofection as described in Example 2, using the same two gRNAs. Equivalent numbers of nucleofected control or Stub1 KO OT-1+CD8+ T cells were introduced intravenously into separate wild-type recipients. A no T cell control was also included which had endogenous T cells but did receive transferred OT-1+ T cells. Five days later, mice were injected subcutaneously with B16 melanoma tumor cells modified to express SIINFEKL (SEQ ID NO:7). Tumor growth was monitored every 2-3 days until mice reached a humane endpoint (poor body condition, tumor exceeded 2000 mm3, or tumor ulcerated). The diameters of the tumor were measured by taking the longest diameter and the perpendicular diameter (x, y axes if facing the mouse). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 25 using the day 25 values and pairwise unpaired t tests.

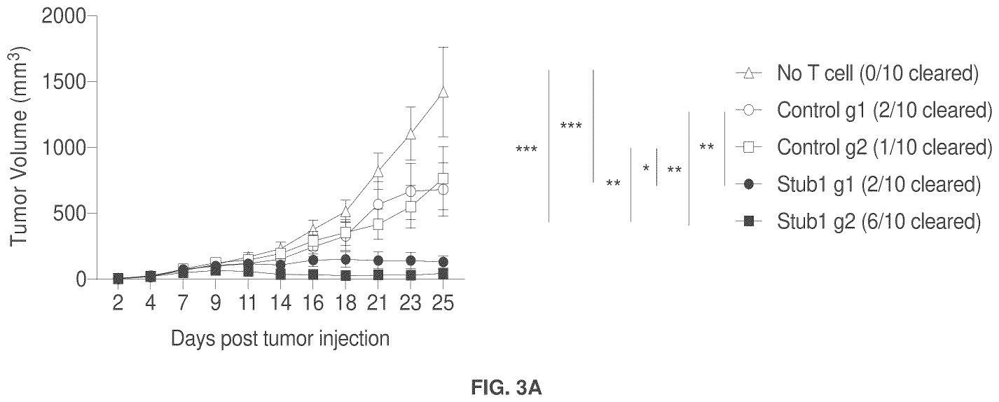

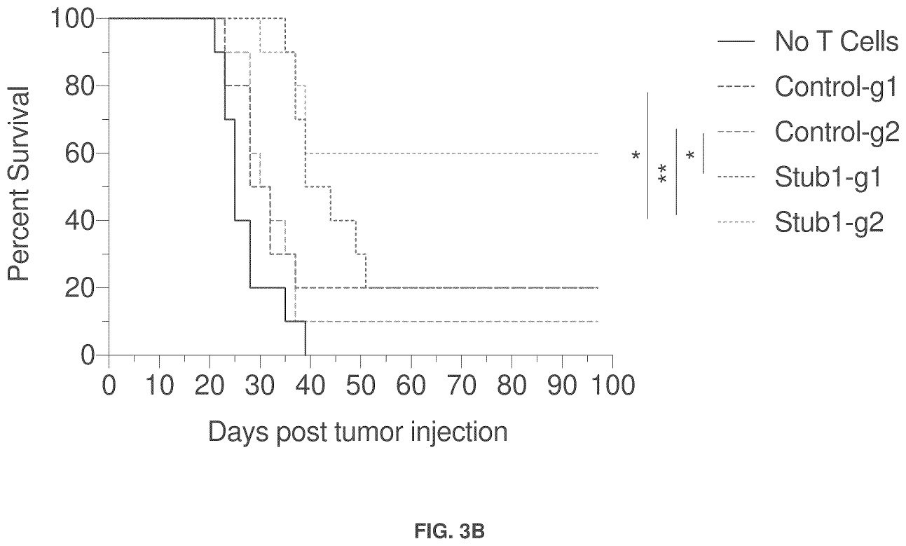

As shown in FIG. 3A, the recipients of Stub1 KO T cells had significantly slower tumor growth than control mice and mice that did not receive OT-1 T cells. In addition, mice receiving Stub1 KO T cells had a longer survival than mice receiving the control T cells (FIG. 3B). Thus, it was demonstrated that Stub1 KO CD8+ T cells control tumors significantly better than control CD8+ T cells.

### Example 4. Assessing Stub1 Knockout (KO) Bone Marrow Chimeras (BMCs) in an In Vivo Tumor Growth Control Assay Shows Significantly Slower Tumor Growth in Stub1KO BMCs than Control BMCs

To examine how Stub1 KO in all hematopoietic cells affects tumor growth, a tumor growth control assay was performed using control and Stub1 KO BMCs. Tumor growth in BMCs that have control or Stub1 KO hematopoietic systems were compared to determine the effect of Stub1 KO on all hematopoietic cells on tumor clearance.

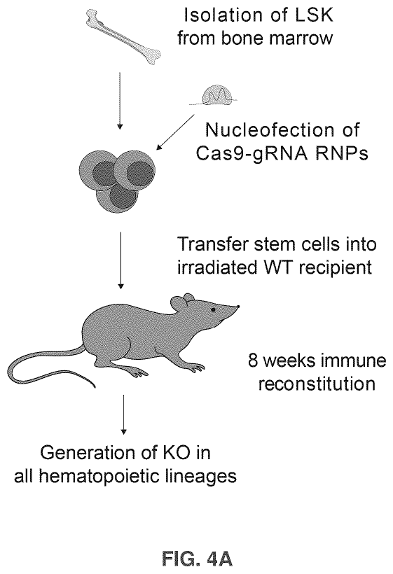

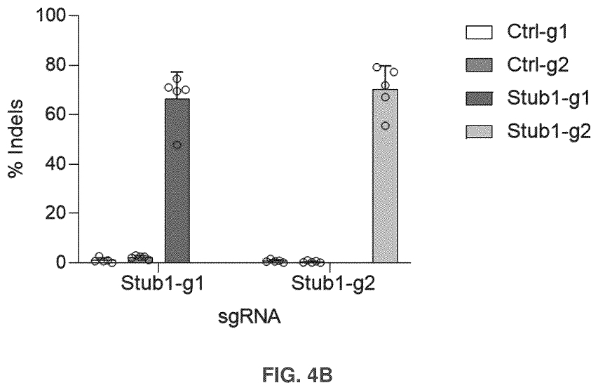

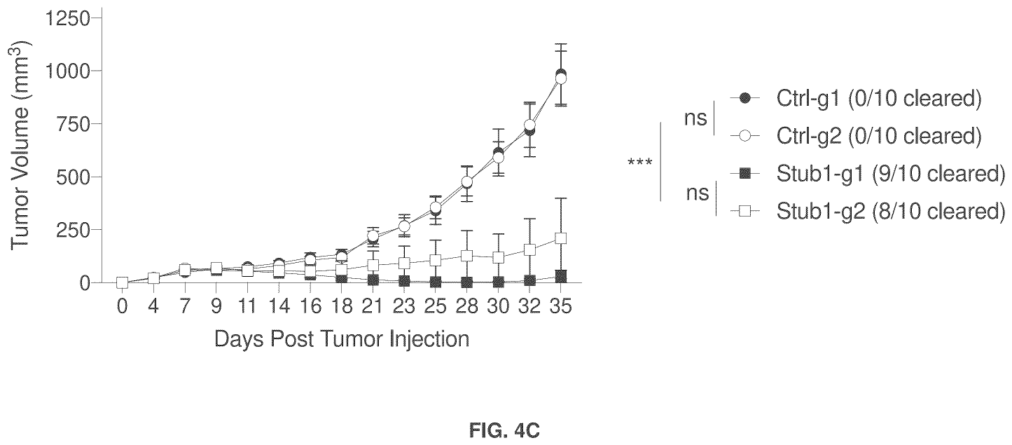

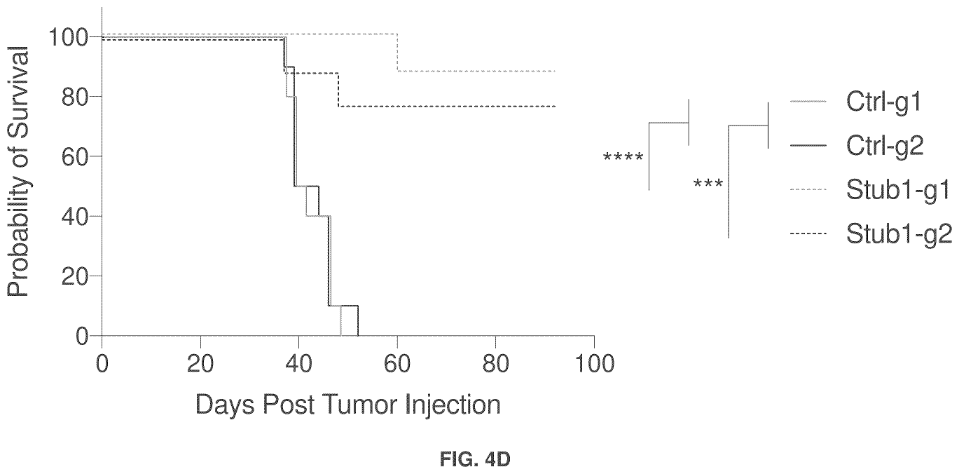

BMCs were prepared by nucleofecting c-Kit+bone marrow cells (from wild-type mice) with control or Stub1 gRNAs complexed with Cas9 (FIG. 4A). The nucleofected bone marrow cells were intravenously transferred into irradiated wild-type recipient mice. These mice were given 8 weeks to reconstitute their immune systems. BMCs were then bled retroorbitally to obtain immune cells, from which the genomic DNA was subsequently isolated. The TIDE assay was performed to assess insertion-deletion (indel) formation in the Stub1 gene. About 1 week later, these BMCs were injected with MC38 (colorectal cancer) cells subcutaneously. Tumor growth was monitored every 2-3 days until mice reached a humane endpoint (poor body condition, tumor exceeded 2000 mm3, or tumor ulcerated). The diameters of the tumor were measured by taking the longest diameter and the perpendicular diameter (x, y axes if facing the tumor). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 35 using the day 35 values in a one-way ANOVA test.

As shown in FIG. 4B, Stub1 was deleted in about 70% of immune cells with each gRNA. The Stub1 KO BMCs showed significantly slower tumor growth than the matched control BMCs (FIG. 4C), suggesting that Stub1 is an attractive target for cancer immunotherapy. In addition, mice receiving Stub1 KO BMC cells had a longer survival than mice receiving the control cells (FIG. 4D). Thus, it was demonstrated that Stub1 KO BMCs significantly attenuates tumor growth compared with control BMCs.

### Example 5. Assessing Stub1 KO CD8+ T Cells in a Competition Assay in the LLC-OVA Tumor Model

In order to assess Stub1 KO CD8+ T cells in a different tumor model (LLC-OVA), a competition assay was used to compare a control and a Stub1 KO T cell in the same mouse, enabling comparison of the two T cell types in the same tissue microenvironment. The two populations start at equivalent ratios and following tumor growth these populations are evaluated to determine if this ratio has changed.

Nucleofection was used to deliver gRNA-Cas9 RNPs targeting a control sequence or Stub1. CD45.1 congenically marked OT-1 T cells were nucleofected with a control gRNA, and CD45.1 and CD45.2 congenically marked T cells were nucleofected with control, Ptpn2, or Stub1 gRNAs. The nucleofected naive CD8+ T cells were cultured in vitro in RPMI media containing fetal bovine serum, penicillin-streptomycin, HEPES, non-essential amino acids, sodium pyruvate, and murine IL-7 for 1 day. The next day, cells were counted and mixed in a 50:50 ratio (CD45.1 control gRNA and CD45.1/.2 control, Ptpn2, or Stub1 gRNAs). These cells were (1) transferred to CD45.2 wild-type recipients intravenously or (2) stained with CD45.1 and CD45.2 to assess the exact ratio of cells (should be close to 50:50). Five days later, mice were injected subcutaneously with LLC tumor cells modified to express SIINFEKL (SEQ ID NO:7) (which is recognized by OT-1+ T cells). Tumor formation and growth were monitored for approximately 10 days. Tumors were isolated and congenically marked transferred cells (CD45.1 and CD45.1/.2) assessed by flow cytometry. The ratio of CD45.1 to CD45.1/.2 at output was compared to the input ratio and the log-normalized fold change calculated. All sample ratios were compared to control vs. control for significance.

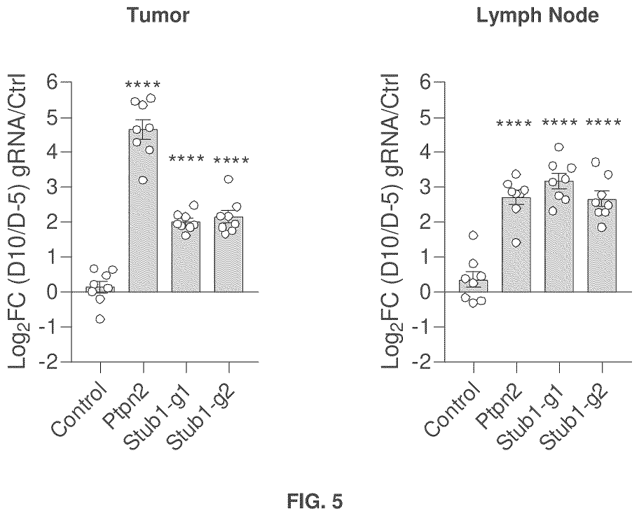

As shown in FIG. 5, both gRNAs for Stub1 were significantly enriched in tumor and lymph nodes when compared to the control gRNA mix. In addition, the positive control Ptpn2 was significantly enriched. These results validated Stub1 as a negative regulator of CD8+ T cell abundance in LLC-OVA tumors, and further demonstrate that Stub1 KO CD8+ T cells are enriched in multiple tumor models (e.g., B16-OVA and LLC-OVA).

### Example 6. Assessing the Proliferation of Stub1 KO CD8+ T Cells In Vitro

In order to assess the proliferation of Stub1 KO CD8+ T cells in vitro, a cell trace violet (CTV) dilution assay was performed. This assay involves initially labeling the cells with a bright fluorescent dye (CTV) and then activating the cells to induce division. As cells divide, the intensity of the dye will decrease proportionally enabling evaluation of the proliferative history of a cell following proliferation. This experiment was performed twice to ensure reproducibility.

Nucleofection was used to deliver gRNA-Cas9 RNPs targeting a control sequence or Stub1. The nucleofected naive CD8+ T cells were cultured in vitro in RPMI media containing fetal bovine serum, penicillin-streptomycin, HEPES, non-essential amino acids, sodium pyruvate, and murine IL-7 for 6 days, to enable dilution of residual Stub1 RNA and protein following CRISPR-mediated editing of the DNA. The control and Stub1-deleted naive CD8+ T cells were then CTV labeled and plated on anti-CD3 and anti-CD28 cross-linking antibodies in RPMI media containing fetal bovine serum, penicillin-streptomycin, HEPES, non-essential amino acids, sodium pyruvate, and murine IL-2 for 3 days. Note anti-CD3, anti-CD28, and IL-2 will activate the T cells, which results in proliferation and dilution of the CTV. CTV dilution was assessed after 3 days of activation by flow cytometry.

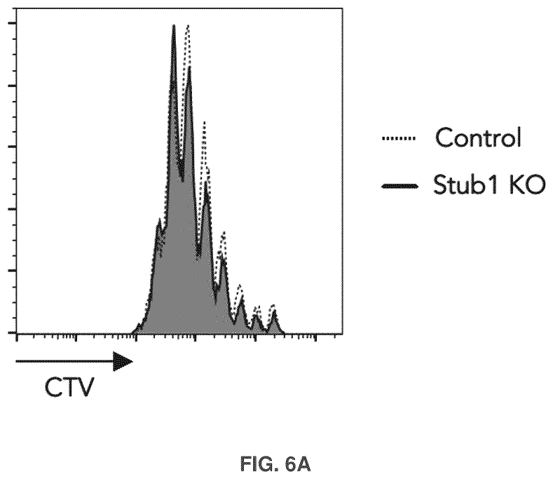

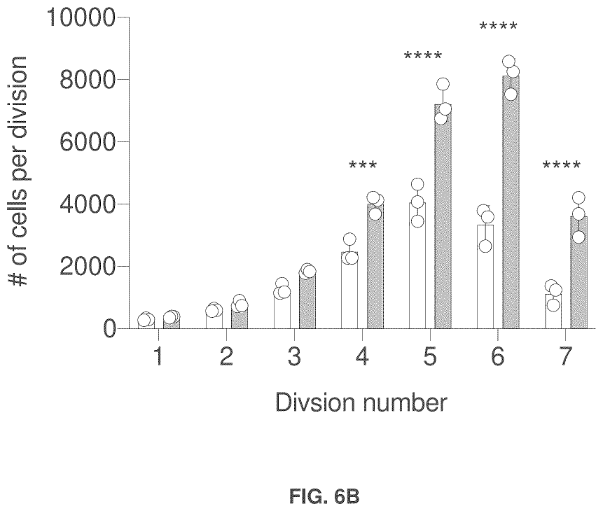

As shown in FIG. 6A and FIG. 6B, Stub1KO CD8+ T cells proliferated significantly more than control CD8+ T cells.

### Example 7. Assessing Expression of Functional Markers by Stub1 KO CD8+ T Cells In Vivo

The functionality of Stub1 KO CD8+ T cells in tumors was assessed by flow cytometry. Nucleofection to deliver gRNA-Cas9 RNPs targeting a control sequence or Stub1. Note CD45.1 congenically marked OT-1 T cells were nucleofected with control or Stub1 gRNAs. The nucleofected naive CD8+ T cells were cultured in vitro in RPMI media containing fetal bovine serum, penicillin-streptomycin, HEPES, non-essential amino acids, sodium pyruvate, and murine IL-7 for 1 day. The next day, cells were counted and transferred to separate groups of CD45.2 wild-type recipients intravenously. Five days later, mice were injected subcutaneously with B16 melanoma cells modified to express SIINFEKL (SEQ ID NO:7) (which is recognized by OT-1+ T cells). Tumor formation and growth were monitored for 9 days. Tumors were isolated and congenically marked transferred cells (CD45.1) assessed by flow cytometry. The cells were stained with PD-1, CD25 (IL2 receptor alpha), Granzyme B (GzmB), Perforin (Pfn), interferon gamma (IFNγ), tumor necrosis factor alpha (TNFα), CD107a (marker of degranulation), Slamf6 (marker of progenitor exhausted T cells), and Tim-3 (marker of terminally exhausted T cells).

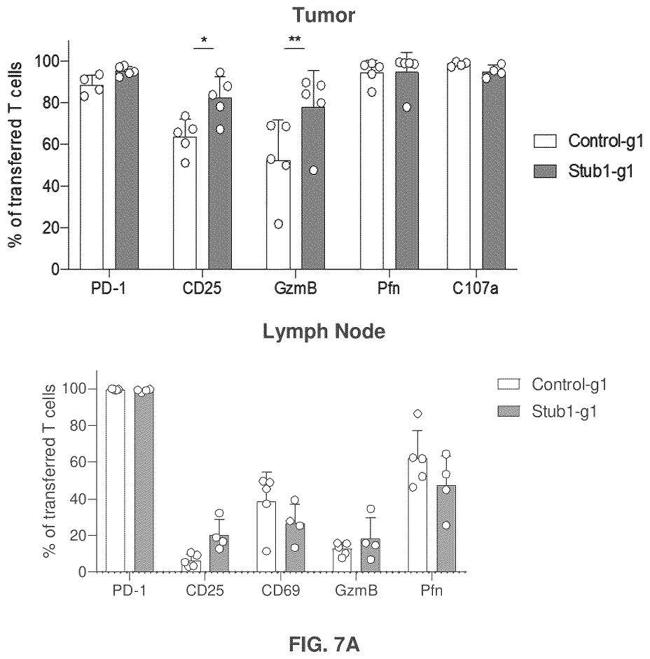

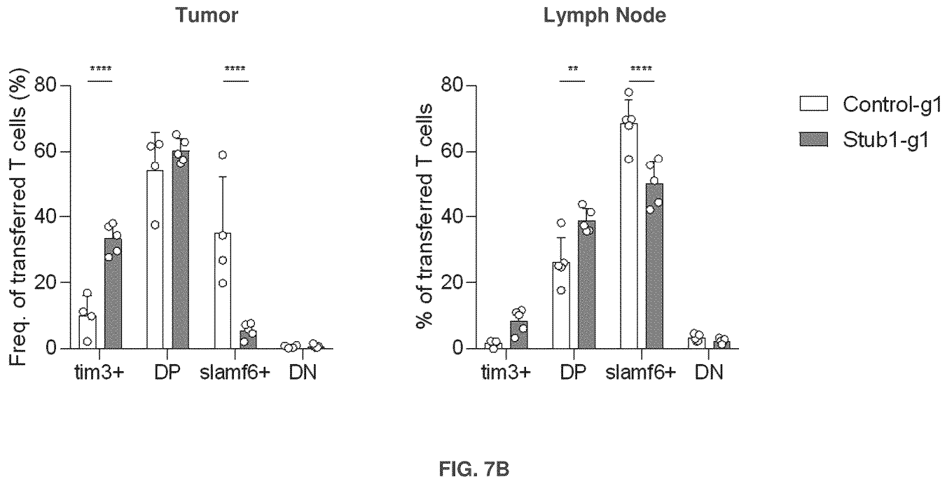

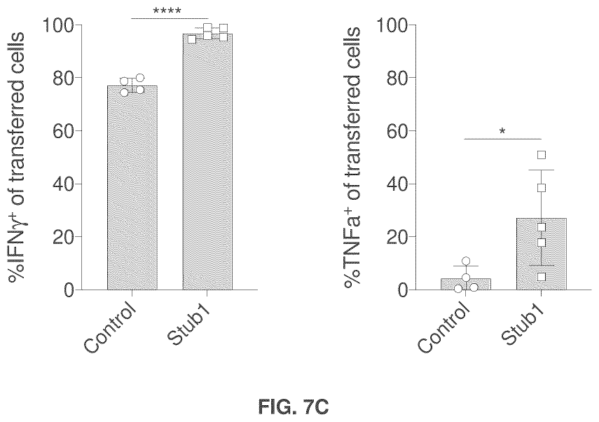

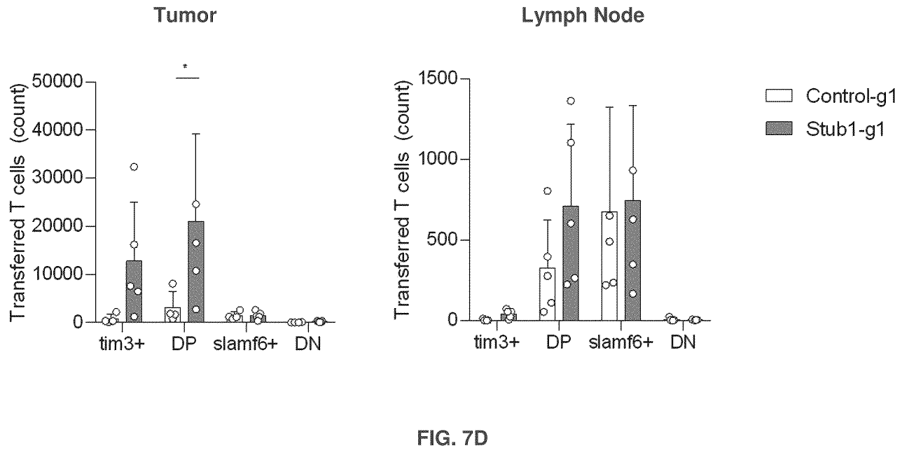

As shown in FIG. 7A and FIG. 7B, Stub1 KO CD8+ T cells express more CD25, granzyme B, and Tim-3 compared to control cells. As shown in FIG. 7C, Stub1KO CD8+ T cells express more IFNγ and TNFα compared to control cells. Stub1 KO CD8+ T cells express less Slamf6 compared to control cells, suggesting that Stub1 KO CD8+ T cells are more activated and cytotoxic. Stub1 KO increases the ratio of terminally exhausted (Slamf6-Tim-3+) to progenitor exhausted (Slamf6+Tim-3−) CD8+ T cells, suggesting that Stub1 controls the formation or maintenance of the terminally and progenitor exhausted subpopulations. By cell count, Stub1 KO CD8+ T cells maintain a subset of Slamf6+progenitor exhausted cells that is not significantly different from control cells (FIG. 7D).

### Example 8. Assessing Stub1 KO CD8+ T Cells in a Competitive Assay in Multiple Organs

The ability of Stub1 KO CD8+ T cells to outcompete control cells in non-tumor organs, such as the tumor-draining lymph node and the spleen, was assessed. This experiment was performed twice to ensure reproducibility.

Nucleofection was used to deliver gRNA-Cas9 RNPs targeting a control sequence or Stub1. CD45.1 congenically marked OT-1 T cells were nucleofected with a control gRNA, and CD45.1 and CD45.2 congenically marked T cells were nucleofected with a Stub1 gRNA. The nucleofected naive CD8+ T cells were cultured in vitro in RPMI media containing fetal bovine serum, penicillin-streptomycin, HEPES, non-essential amino acids, sodium pyruvate, and murine IL-7 for 1 day. The next day, cells were counted and mixed in a 50:50 ratio (CD45.1 control gRNA and CD45.1/.2 Stub1 gRNA). These cells were (1) transferred to CD45.2 wild-type recipients intravenously or (2) stained with CD45.1 and CD45.2 to assess the exact ratio of cells (should be close to 50:50). Five days later, mice were injected subcutaneously with B16 melanoma cells modified to express SIINFEKL (SEQ ID NO:7) (which is recognized by OT-1+ T cells). Tumor formation and growth were monitored for approximately 10 days. Tumors were isolated and congenically marked transferred cells (CD45.1 and CD45.1/.2) assessed by flow cytometry. The ratio of CD45.1 to CD45.1/.2 at output was compared to the input ratio and the log-normalized fold change calculated.

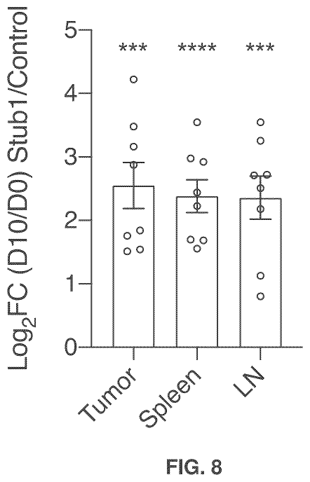

FIG. 8 shows that Stub1 KO CD8+ T cells were significantly enriched compared to our control cells in the tumor, tumor-draining lymph node, and spleen. Stub1 KO CD8+ T cells outcompete control cells in multiple organs. This suggests that Stub1 KO CD8+ T cells are not enriched in tumors merely due to more effective trafficking. Furthermore, the advantage of Stub1 KO CD8+ T cells in the lymph node and spleen suggests that Stub1 may have a role in affecting T cell priming and activation. This is notable as PD-1, which was originally thought to impact CD8+ T cells primarily in tumors, has also recently been shown to modulate T cell priming in tumor-draining lymph nodes.

### Example 9. Assessing Stub1 KO CD8+ T Cells in a Competition Assay in the B16-OVA Tumor Model Following Treatment with PD-1 Blockade

Stub1 KO CD8+ T cells were assessed in a competitive assay following treatment with PD-1 blockade or isotype control antibody. A competition assay was used to compare a control and a Stub1 KO T cell in the same mouse, enabling comparison of the two T cell types in the same tissue microenvironment. The two populations start at equivalent ratios and following tumor growth these populations are evaluated to determine if this ratio has changed. This experiment was performed twice to ensure reproducibility.

Nucleofection was used to deliver gRNA-Cas9 RNPs targeting a control sequence or Stub1. CD45.1 congenically marked OT-1 T cells were nucleofected with a control gRNA, and CD45.1 and CD45.2 congenically marked T cells were nucleofected with a Stub1 gRNA. The nucleofected naive CD8+ T cells were cultured in vitro in RPMI media containing fetal bovine serum, penicillin-streptomycin, HEPES, non-essential amino acids, sodium pyruvate, and murine IL-7 for 1 day. The next day, cells were counted and mixed in a 50:50 ratio (CD45.1 control gRNA and CD45.1/.2 control or Stub1 gRNAs). These cells were (1) transferred to CD45.2 wild-type recipients intravenously or (2) stained with CD45.1 and CD45.2 to assess the exact ratio of cells (should be close to 50:50). Five days later, mice were injected subcutaneously with B16 tumor cells modified to express SIINFEKL (SEQ ID NO:7) (which is recognized by OT-1+ T cells). Tumor formation and growth were monitored for approximately 9 days. Nine days post tumor injection, mice were administered 5 mg/kg isotype control antibody or anti-PD-1 blocking antibody. Note the PD-1 blocking antibody will remain bound to PD-1 for 5 days in vivo at this dosage and thus only one treatment was required for this experiment. Thirteen days post tumor injection, tumors were isolated and congenically marked transferred cells (CD45.1 and CD45.1/.2) assessed by flow cytometry. The ratio of CD45.1 to CD45.1/.2 at output was compared to the input ratio and the log-normalized fold change calculated.

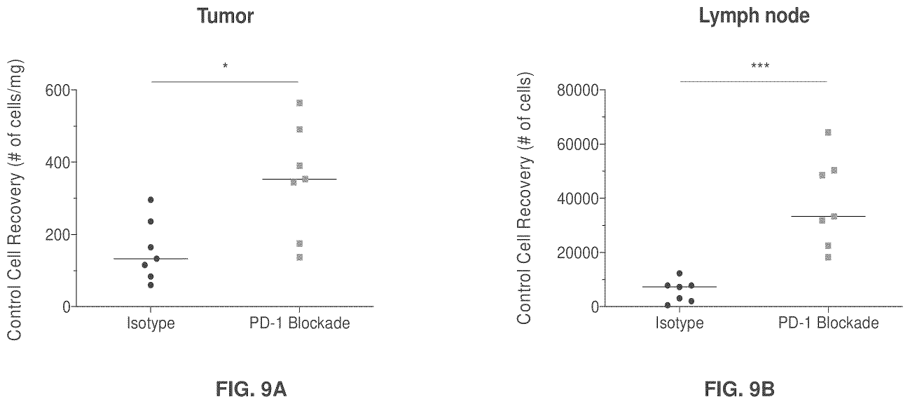

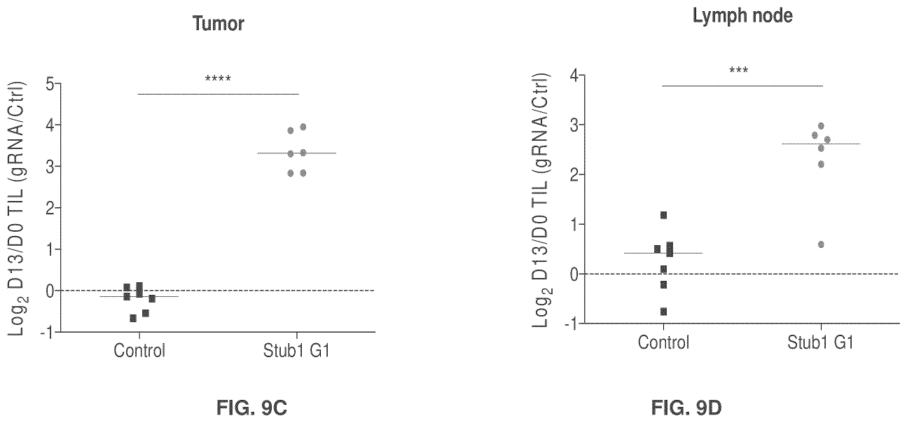

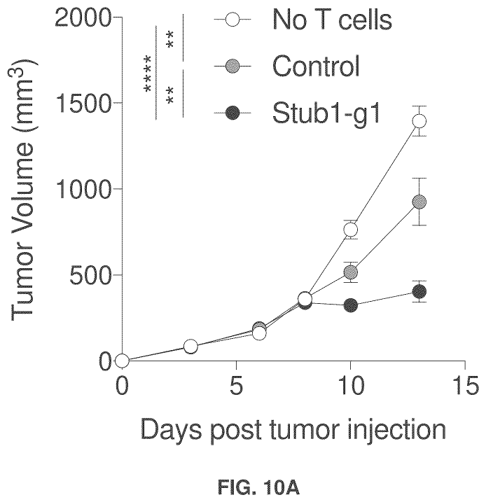

As shown in FIG. 9A and FIG. 9B, PD-1 blockade treatment significantly increased the number of control cells in tumors and in lymph nodes when compared with isotype control treatment. This suggests that PD-1 blockade is able to increase cell numbers in tumors and in lymph nodes in the timeframe of this experiment.

As shown in FIG. 9C and FIG. 9D, Stub1 KO cells were significantly enriched following PD-1 blockade treatment in tumors and in lymph nodes when compared to control cells. This suggests that PD-1 blocked Stub1KO cells can outcompete PD-1 blocked control cells in tumors and in lymph nodes.

In conclusion, Stub1 KO CD8+ T cells outcompete control cells following PD-1 blockade treatment in tumors and tumor-draining lymph nodes. Thus, Stub1 KO provides CD8+ T cells with an additional advantage beyond the advantage provided by PD-1 blockade treatment alone.

### Example 10. Assessing Stub1 Knockout (KO) T Cells in an In Vivo Tumor Growth Control Assay Shows Stub1 KO CD8+ T Cells Control Tumors Significantly Better than Control CD8+ T Cells

To determine whether Stub1 KO CD8+ T cells control tumor growth more effectively than control CD8+ T cells, a tumor growth control assay was performed using control and Stub1 KO T cells. For this experiment, control and Stub1 KO OT-1+ T cell populations were transferred into separate mice. These mice were injected with Lewis lung carcinoma tumor cells expressing SIINFEKL (which is recognized by OT-1+ T cells), and tumor growth was monitored. Tumor growth in mice with Stub1 KO OT-1+ T cells was compared with mice receiving the control cells.

Stub1 KO naive OT-1+CD8+ T cells and control cells were created using nucleofection as described in Example 2. Equivalent numbers of nucleofected control or Stub1 KO OT-1+CD8+ T cells were introduced intravenously into separate wild-type recipients. A no T cell control was also included which had endogenous T cells but did receive transferred OT-1+ T cells. Five days later, mice were injected subcutaneously with LLC tumor cells modified to express SIINFEKL. Tumor growth was monitored every 2-3 days until mice reached a humane endpoint (poor body condition, tumor exceeded 2000 mm³, or tumor ulcerated). The diameters of the tumor were measured by taking the longest diameter and the perpendicular diameter (x, y axes if facing the mouse). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 13 using the day 13 values and a one-way ANOVA test.

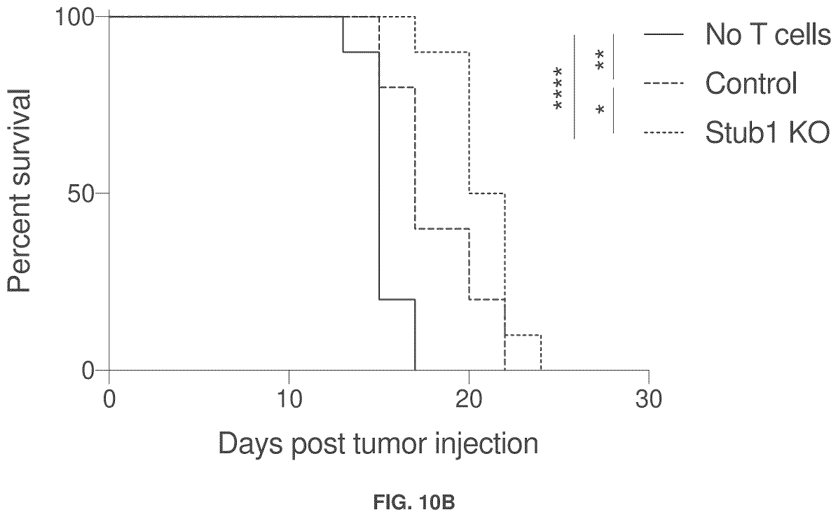

As shown in FIG. 10A, the recipients of Stub1 KO T cells had significantly slower tumor growth than control mice and mice that did not receive OT-1 T cells. In addition, mice receiving Stub1 KO T cells had a longer survival than mice receiving the control T cells (FIG. 10B). Thus, it was demonstrated that Stub1 KO CD8+ T cells control LLC-OVA tumors significantly better than control CD8+ T cells.

### Example 11. Assessing Stub1 Knockout (KO) Bone Marrow Chimeras (BMCs) in an In Vivo Tumor Growth Control Assay Shows Significantly Slower Tumor Growth in Stub1KO BMCs than Control BMCs

To examine how Stub1 KO in all hematopoietic cells affects tumor growth, a tumor growth control assay was performed using control and Stub1 KO BMCs. Tumor growth in BMCs that have control or Stub1 KO hematopoietic systems was compared to determine the effect of Stub1 KO on all hematopoietic cells on tumor clearance.

BMCs were prepared by nucleofecting c-Kit+bone marrow cells (from wild-type mice) with control or Stub1 gRNAs complexed with Cas9. The nucleofected bone marrow cells were intravenously transferred into irradiated wild-type recipient mice. These mice were given 8 weeks to reconstitute their immune systems. BMCs were then bled retroorbitally to obtain immune cells, from which the genomic DNA was subsequently isolated. The TIDE assay was performed to assess insertion-deletion (indel) formation in the Stub1 gene. About 1 week later, these BMCs were injected with B16 melanoma cells expressing SIINFEKL (B16-OVA) subcutaneously. Tumor growth was monitored every 2-3 days until mice reached a humane endpoint (poor body condition, tumor exceeded 2000 mm³, or tumor ulcerated). The diameters of the tumor were measured by taking the longest diameter and the perpendicular diameter (x, y axes if facing the tumor). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 27 using the day 27 values in a one-way ANOVA test.

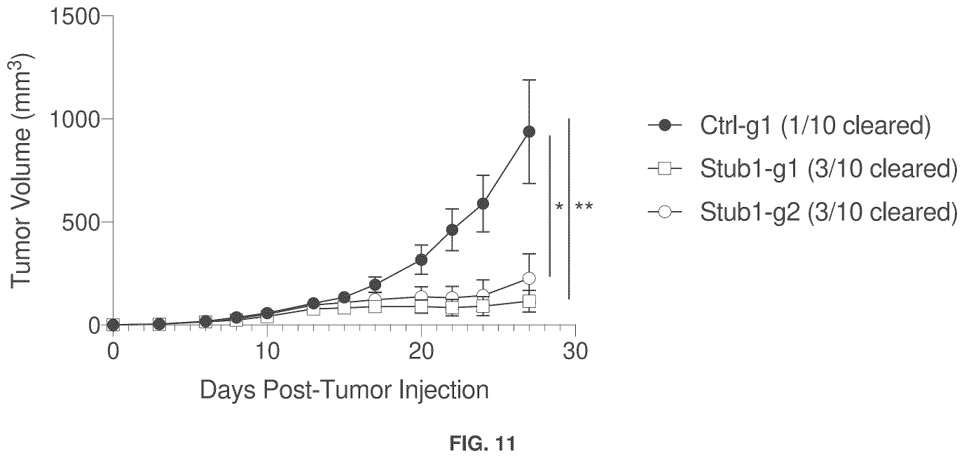

As shown in FIG. 11, the Stub1 KO BMCs showed significantly slower B16-OVA tumor growth than the matched control BMCs.

### Example 12. Stub1 KO BMC Tumor Growth Control in the MC38 Model with CD8 Depletion or Isotype Control Shows CD8-Dependence of Phenotype

To examine if the MC38 tumor growth control displayed by Stub1 KO BMCs was dependent on CD8+ T cells, MC38 tumor growth in BMCs that have control or Stub1 KO hematopoietic systems and were depleted of CD8+ T cells (or treated with an isotype control) were compared.

BMCs were prepared by nucleofecting c-Kit+bone marrow cells (from wild-type mice) with control or Stub1 gRNAs complexed with Cas9. The nucleofected bone marrow cells were intravenously transferred into irradiated wild-type recipient mice. These mice were given 8 weeks to reconstitute their immune systems. BMCs were then bled retroorbitally to obtain immune cells, from which the genomic DNA was subsequently isolated. The TIDE assay was performed to assess insertion-deletion (indel) formation in the Stub1 gene. These BMCs were then treated with CD8-depleting antibodies or isotype control antibodies every 3 days for the duration of the experiment. About 1 week later, these BMCs were injected with MC38 colorectal cancer cells subcutaneously. Tumor growth was monitored every 2-3 days until mice reached a humane endpoint (poor body condition, tumor exceeded 2000 mm³, or tumor ulcerated). The diameters of the tumor were measured by taking the longest diameter and the perpendicular diameter (x, y axes if facing the tumor). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 30 using the day 30 values in a one-way ANOVA test.

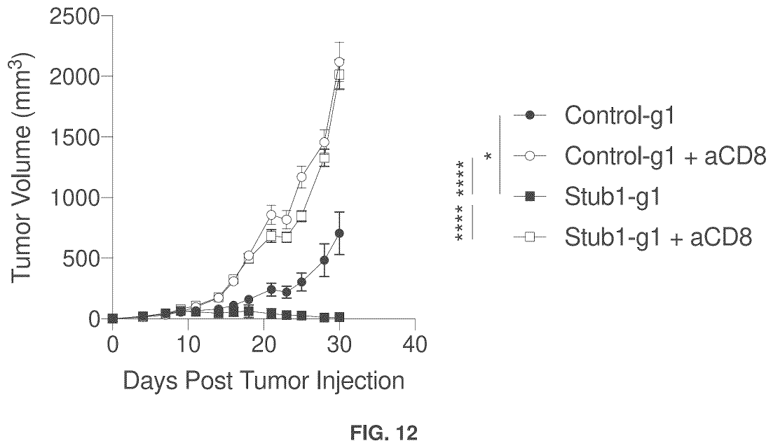

As shown in FIG. 12, the control of MC38 tumors by Stub1 KO BMCs depends on the presence of CD8+ T cells.

### Example 13. Stub1 KO on a WT or IFNγ KO Background in the B16-OVA Model Shows Dependence of IFNγ for Improved Tumor Control by Stub1 KO T Cells

To determine whether Stub1 KO CD8+ T cells require IFNg to control tumor growth, a tumor growth control assay was performed using control and Stub1 KO T cells on either a wild-type background or an IFNg KO background. For this experiment, control and Stub1 KO OT-1+ T cell populations were transferred into separate mice. These mice were injected with B16 melanoma tumor cells expressing SIINFEKL (which is recognized by OT-1+ T cells), and tumor growth was monitored.

Stub1 KO naive OT-1+CD8+ T cells and control cells were created using nucleofection. Equivalent numbers of nucleofected OT-1+CD8+ T cells were introduced intravenously into separate wild-type recipients. Five days later, mice were injected subcutaneously with B16 tumor cells modified to express SIINFEKL. Tumor growth was monitored every 2-3 days until mice reached a humane endpoint (poor body condition, tumor exceeded 2000 mm³, or tumor ulcerated). The diameters of the tumor were measured by taking the longest diameter and the perpendicular diameter (x, y axes if facing the mouse). The z dimension was assumed to be equivalent to the shorter tumor diameter as is standard in the literature. These values were used to calculate the tumor volume using the formula for calculating the volume of an ellipsoid. Tumor growth was plotted according to volume over time. Significance was calculated at day 17 using the day 17 values and a one-way ANOVA test.

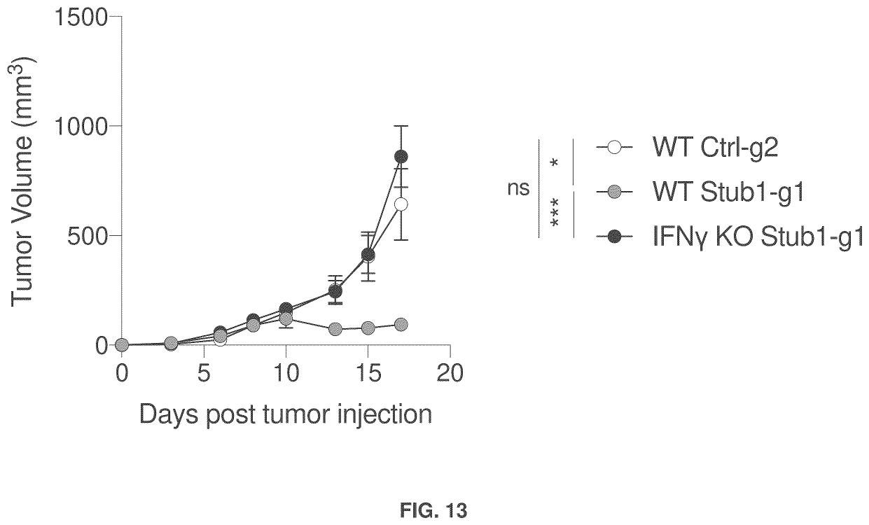

As shown in FIG. 13, the recipients of Stub1 KO T cells had significantly slower tumor growth than control mice. In addition, mice receiving Stub1 KO IFNg KO T cells did not significantly differ from control T cells. Thus, IFNg is required for the increased tumor growth control capacity of Stub1 KO T cells.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of treating a cancer in a subject in need thereof, comprising decreasing the expression and/or activity of STIP1 Homology and U-Box Containing Protein 1 (Stub1) in an immune cell of the subject, thereby treating the cancer in the subject.

2. The method of claim 1, wherein the immune cell is selected from the group consisting of a T cell, a B cell, a natural killer (NK) cell, a dendritic cell, and a macrophage.

3. (canceled)

4. The method of claim 1, wherein the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

5. The method of claim 4,
(a) wherein the agent is a modified immune cell;
(b) wherein the agent is a small molecule inhibitor of Stub1; and/or
(c) wherein the agent inhibits interaction between Stub1 and a binding partner; optionally, wherein the binding partner is cysteine-rich hydrophobic domain 2 (Chic2).

6. The method of claim 4,
(a) wherein the modified immune cell is an immune cell derived from the subject modified to have a decreased level of expression and/or activity of Stub1;
(b) wherein the modified immune cell is a modified T cell having a decreased level of expression and/or activity of Stub1;
(c) wherein the modified immune cell comprises a STUB1 knockout T cell;
(d) wherein the modified immune cell further comprises a nucleic acid encoding a chimeric antigen receptor (CAR), wherein the CAR comprises an antigen binding domain, a transmembrane domain and an intracellular domain of a co-stimulatory molecule; and/or
(e) wherein the modified immune cell comprises a STUB1 knockout CAR T cell.

7-11. (canceled)

12. The method of claim 1, wherein the subject is a human subject.

13. The method of claim 1, wherein the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

14. A method of increasing an immune response against a tumor and/or reducing a tumor size in a subject in need thereof, comprising decreasing the expression and/or activity of Stub1 in an immune cell of the subject, thereby increasing an immune response against the tumor and/or reducing the tumor size in the subject.

15. The method of claim 14, wherein the immune cell is selected from the group consisting of a T cell, a B cell, a natural killer (NK) cell, a dendritic cell, and a macrophage.

16. (canceled)

17. The method of claim 14,
(a) wherein the method increases a T cell response;
(b) wherein the method increases the number and/or activity of T cells;
(c) wherein the method increases the expression of at least one marker selected from the group consisting of CD25, Granzyme B, TNFα, IFNγ, and Tim-3 in the immune cell; and/or
(d) wherein the method decreases the percentage of Slamf6-expressing immune cells and/or expression of Slamf6 in the immune cell.

18-21. (canceled)

22. The method of claim 14, wherein the expression and/or activity of STUB1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

23. The method of claim 22,
(a) wherein the agent is a modified immune cell;
(b) wherein the agent is a small molecule inhibitor of Stub1; and/or
(c) wherein the agent inhibits interaction between Stub1 and a binding partner; optionally, wherein the binding partner is cysteine-rich hydrophobic domain 2 (Chic2).

24. The method of claim 22,
(a) wherein the modified immune cell is an immune cell derived from the subject modified to have a decreased level of expression and/or activity of Stub1;
(b) wherein the modified immune cell is a modified T cell having a decreased level of expression and/or activity of Stub1;
(c) wherein the modified immune cell comprises a STUB1 knockout T cell;
(d) wherein the modified immune cell further comprises a nucleic acid encoding a chimeric antigen receptor (CAR), wherein the CAR comprises an antigen binding domain, a transmembrane domain and an intracellular domain of a co-stimulatory molecule; and/or
(e) wherein the modified immune cell comprises a STUB1 knockout CAR T cell.

25-30. (canceled)

31. The method of claim 14, wherein the tumor is associated with a cancer selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

32-44. (canceled)

45. A method of prolonging the survival of a subject having a cancer, comprising decreasing the expression and/or activity of Stub1 in an immune cell of the subject, thereby prolonging the survival of the subject.

46. The method of claim 45, wherein the immune cell is selected from the group consisting of a T cell, a B cell, a natural killer (NK) cell, a dendritic cell, and a macrophage.

47. (canceled)

48. The method of claim 45, wherein the expression and/or activity of Stub1 is decreased by administration to the subject of an effective amount of at least one agent selected from the group consisting of a small molecule, a modified immune cell, a modified hematopoietic cell, an anti-Stub1 antibody or antigen-binding fragment thereof, an antisense agent targeting STUB1, a double stranded RNA agent targeting STUB1, an RNA-guided nuclease targeting STUB1, a Stub1 fusion protein; and a Stub1 inhibitory peptide.

49. The method of claim 48,
(a) wherein the agent is a modified immune cell;
(b) wherein the agent is a small molecule inhibitor of Stub1; and/or
(c) wherein the agent inhibits interaction between Stub1 and a binding partner; optionally, wherein the binding partner is cysteine-rich hydrophobic domain 2 (Chic2).

50. The method of claim 48,
(a) wherein the modified immune cell is an immune cell derived from the subject modified to have a decreased level of expression and/or activity of Stub1;
(b) wherein the modified immune cell is a modified T cell having a decreased level of expression and/or activity of Stub1;
(c) wherein the modified immune cell comprises a STUB1 knockout T cell;
(d) wherein the modified immune cell further comprises a nucleic acid encoding a chimeric antigen receptor (CAR), wherein the CAR comprises an antigen binding domain, a transmembrane domain and an intracellular domain of a co-stimulatory molecule; and/or
(e) wherein the modified immune cell comprises a STUB1 knockout CAR T cell.

51-56. (canceled)

57. The method of claim 45, wherein the cancer is selected from the group consisting of breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

58. (canceled)

59. The method of claim 1, further comprising administering to the subject an additional therapeutic treatment.

60-62. (canceled)

63. A method for identifying a compound useful for treating a cancer and/or increasing an immune response against a tumor in a subject, comprising providing a test compound;
determining the effect of the test compound on the expression and/or activity of Stub1 in an immune cell of the subject; and
selecting a compound which decreases the expression and/or activity of Stub1, thereby identifying a compound useful for treating a cancer and/or increasing an immune response against the tumor in the subject.

64. (canceled)
